(19) United States
(12) Patent Application Publication
(10) Pub. No.: US 2018/0160713 A1
(43) Pub. Date: Jun. 14, 2018

(54) METHODS OF SCREENING

(71) Applicant: OPTIBIOTIX LIMITED, Heslington (GB)

(72) Inventors: Stephen Patrick O'Hara, York (GB); Sofia Kolida, York (GB)

(21) Appl. No.: 15/577,302

(22) PCT Filed: May 25, 2016
(86) PCT No.: PCT/GB2016/051515

§ 371 Date: Nov. 27, 2017

(30) Foreign Application Priority Data
May 27, 2015 (GB) 1509021.0

(51) Int. Cl. A23L 33/135; C12Q 1/02; G01N 33/569; A23L 33/21

(52) U.S. Cl. CPC A23L 33/135; C12Q 1/02; G01N 33/56944; G01N 2333/335; A23V 2002/00; A23V 2200/3202; G01N 2333/315; A23L 33/21

(57) ABSTRACT

The present invention relates to a discovery platform for screening one or more Streptococcus, Lactobacillus or Propionibacterium bacterial strains for the ability to produce and/or produce a high yield of galactooligosaccharides (GOS) comprising assessing the β-galactosidase activity of a strain under growth conditions and identifying whether the activity has: a) Miller Unit which are equal to or greater than about 60 for Streptococcus or Lactobacillus strains; or b) Miller Unit which are equal to or greater than about 3 for Propionibacterium strains. The present invention also relates to compositions incorporating GOS produced from bacterial strains identified by the screening process.

### TECHNICAL FIELD OF THE INVENTION

The invention relates to a screening method for predicting and identifying bacterial strains capable of producing high yields of galactooligosaccharides (GOS), by reverse enzyme reaction of β-galactosidases. The resultant GOS can be formulated as a selective prebiotic for the growth of a selected bacterial strain, species or genus.

### BACKGROUND TO THE INVENTION

Probiotics are bacteria which confer health benefits to a host. Typically, cultures of probiotic bacterial strains are consumed or administered to individuals in order to add to and augment the naturally occurring bacteria population of the gut. A number of health benefits have been associated with probiotics, including reducing the incidence of cancer, traveler's diarrhoea, irritable bowel syndrome, and lactose intolerance to name a few. Preliminary studies also indicate that probiotics can be useful in reducing serum levels of cholesterol and blood pressure and help modulate diabetes.

Prebiotics are dietary ingredients which can selectively enhance the numbers and/or activity of beneficial indigenous gut microbiota, such as lactobacilli or bifidobacteria, and are finding much increased application in the food sector. Prebiotics are non digestible food ingredients that are selectively metabolised by colonic bacteria which contribute to improved health. As such, their use can promote beneficial changes within the indigenous gut microbial milieu and they can therefore help survivability of probiotics. They are distinct from most dietary fibres like pectin, celluloses, xylan, which are not selectively metabolised in the gut. Criteria for classification as a prebiotic is that it must resist gastric acidity, hydrolysis by mammalian enzymes and absorption in the upper gastrointestinal tract, it is fermented by intestinal microflora and selectively stimulates the growth and/or activity of intestinal bacteria associated with health and well-being.

Fructo-oligosaccharides (FOS, inulin and oligofructose) and galactooligosaccharides (GOS) have been demonstrated to fulfil the criteria for prebiotic classification repeatedly in human intervention studies.

It is an object of the present invention to provide a method of predicting the likelihood a bacterial strain has of being able to produce prebiotics in relatively high yields. It is also an object of the present invention to provide a screening method for quickly identifying probiotic bacterial strains which are capable of producing and/or producing a high yield of GOS which could in turn be used as a selective growth prebiotic for that particular strain, species or genus. It would be advantageous if the screening method was high-through put.

### SUMMARY OF THE INVENTION

In accordance with a first aspect of the present invention, there is provided a method of screening one or more Streptococcus, Lactobacillus or Propionibacterium bacterial strains for the ability to produce and/or produce a high yield of galactooligosaccharides (GOS) comprising assessing the β-galactosidase activity of a strain under growth conditions and identifying whether the activity has:
a) Miller Units which are equal to or greater than about 60 for Streptococcus or Lactobacillus strains; or
b) Miller Units which are equal to or greater than about 3 for Propionibacterium strains.

The method may comprise growing the one or more strains under standard growth conditions for a given incubation time and then lysing the cells and assessing the β-galactosidase activity in the lysate.

The method may further comprise:
i) incubating the one or more strains at about 37° C. for up to 40 hours;
ii) centrifuging the cells at a lower temperature than during incubation;
iii) lysing the cells and removing a supernatant from the lysed cells; and
iv) assessing β-galactosidase activity, expressed in Miller Units, in the supernatant.

If more than one strains are identified by the method as having the required β-galactosidase activity, the method may further comprise:

c) screening the strains at higher and lower temperatures (at least one of which will be different to the growth temperature) at a number of time points to assess which strains have the highest yield of GOS. The higher temperature may be about 50° C. and the lower temperature may be about 30° C.

The bacterial strains may comprise strains selected from: Lactobacillus acidophilus, Lactobacillus reuteri, Lactobacillus rhamnosus, Lactobacillus plantarum, Lactobacillus delbrueckii, Lactobacillus fermentum, Lactobacillus crispatus, Lactobacillus buchneri Lactobacillus helveticus, Streptococcus thermophilus, Propionibacterium jensenii; Propionibacterium freudenreichii; Propionibacterium acidipropionici, or sub-species or mutant strain thereof.

In accordance with another related aspect, there is provided a method of screening a multiplicity of bacterial strains to identify a bacterial strain or strains, which would be suitable for high yield production of a prebiotic composition, the method comprising assessing the growth rate, enzyme production and enzyme activity of an enzyme utilised for the generation of the prebiotic composition by the bacterial strain for each strain and selecting those strains showing the highest growth rate, enzyme production and enzyme activities.

In accordance with a further aspect of the present invention, there is provided a prebiotic composition comprising a galactooligosaccharide (GOS) produced from one or more Streptococcus, Lactobacillus or Propionibacterium bacterial strains, wherein the GOS acts as a selective growth medium for the Streptococcus, Lactobacillus or Propionibacterium bacterial strains, the GOS being in substantially the same form as produced by reverse β-galactosidase reaction in the bacterial strains and the β-galactosidase activity of the Streptococcus, Lactobacillus or Propionibacterium bacterial strains having:
a) a Miller Unit which is equal to or greater than about 60 for Streptococcus or Lactobacillus strains; or
b) a Miller Unit which is equal to or greater than about 3 for Propionibacterium strains.

The GOS may be produced and/or is selective for one of more of the following bacterial strains: Lactobacillus acidophilus, Lactobacillus reuteri, Lactobacillus rhamnosus, Lactobacillus plantarum, Lactobacillus delbrueckii, Lactobacillus fermentum, Lactobacillus crispatus, Lactobacillus buchneri Lactobacillus helveticus, Streptococcus thermophilus, Propionibacterium jensenii; Propionibacterium freudenreichii; Propionibacterium acidipropionici, or sub-species or mutant strain thereof.

The prebiotic composition will preferably be present in the composition in an effective amount so as to elicit a positive and gradual change in the proportions of Lactobacillus or Propionibacterium probiotic bacterial strains in the gut. Higher amounts may be utilised if change in the microbiota is required quickly or if the composition is being used to help seed the gut with a new bacterial strain not currently present.

The prebiotic composition may be encapsulated. Many encapsulation techniques will be apparent to the skilled addressee and the one employed will be tailored to the required stability of the prebiotic growth medium during digestive transit.

The prebiotic composition may further comprise an excipient or carrier compound to enable it to pass through at least part of the gastrointestinal environment of the body and be efficiently delivered to, and released in the lower gut. The prebiotic may be concentrated and/or freeze dried. The composition may be in a number of formats, such as in the form of a liquid (which may be drinkable) and/or powder which can be mixed with a solid or liquid food stuff.

The prebiotic composition may be combined with one or more active ingredients, such as vitamins, minerals, phytochemicals, antioxidants, probiotic bacterial strains and combinations thereof.

Vitamins may include fat soluble vitamins such as vitamin A, vitamin D, vitamin E, and vitamin and combinations thereof. In some embodiments, vitamins can include water soluble vitamins such as vitamin C (ascorbic acid), the B vitamins (thiamine or B1, riboflavin or B25 niacin or B3, pyridoxine or B6, folic acid or B9, cyanocobalamin or B12, pantothenic acid, biotin), and combinations thereof.

Minerals may include but are not limited to sodium, magnesium, chromium, iodine, iron, manganese, calcium, copper, fluoride, potassium, phosphorous, molybdenum, selenium, zinc, and combinations thereof.

Antioxidants may include but are not limited to ascorbic acid, citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, flavonoids, and combinations thereof.

Phytochemicals may include but are not limited to cartotenoids, chlorophyll, chlorophyllin, fiber, flavanoids, anthocyanins, cyanidin, delphinidin, malvidin, pelargonidin, peonidin, petunidin, flavanols, catechin, epicatechin, epigallocatechin, epigailocatechin gallate, theaflavins, thearubigins, proanthocyanins, flavonols, quercetin, kaempferol, myricetin, isorhamnetin, hesperetin, naringenin, eriodictyol, tangeretin, flavones, apigenin, luteolin, lignans, phytoestrogens, resveratrol, isoflavones, daidzein, genistein, glycitein, soy isoflavones, and combinations thereof.

The composition may be for use as a medicament and/or a dietary supplement and/or a nutraceutical or a functional food.

Preferably, the GOS of the composition is produced by a strain or strains identified in the screening method as herein above described.

### DETAILED DESCRIPTION OF THE INVENTION

Embodiments of the present invention will now be described, by way of example only and with reference to the following Figures:

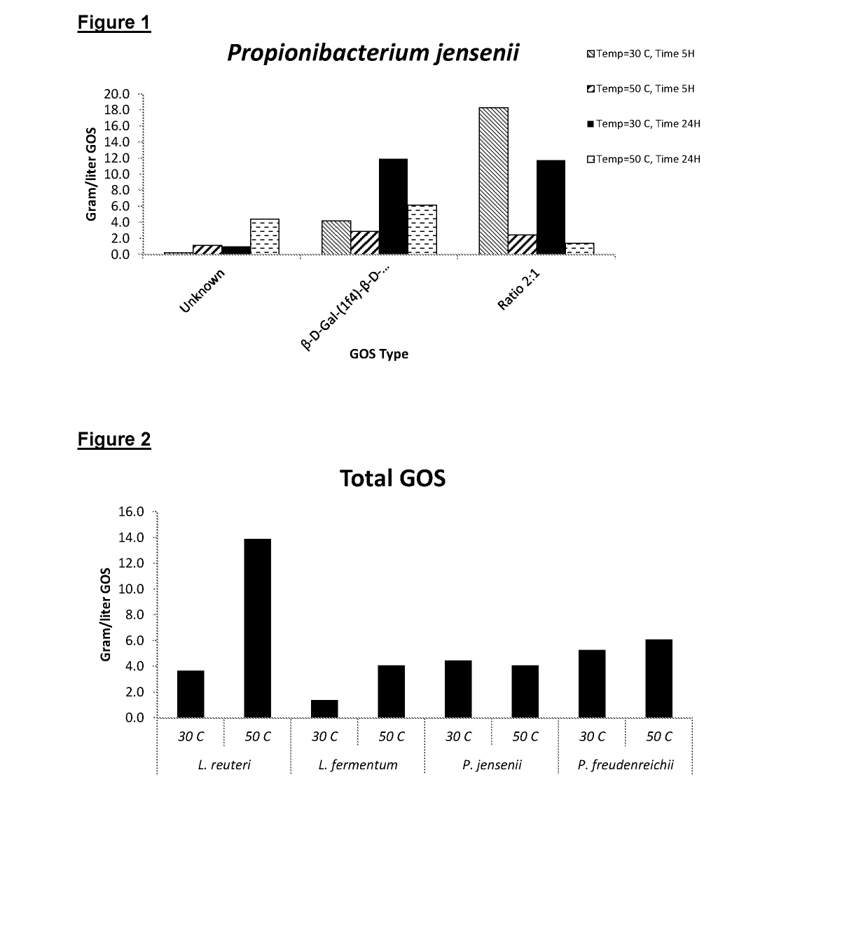

FIG. 1 is a graph showing the ratio between the most prevalent GOS species for P. jensenii synthesized at different temperatures and timepoints;

FIG. 2 is a graph showing the two GOS formation rates at 30° C. and 50° C. of the selected Lactobacillus and two Propionibacterium strains;

FIG. 3 is a graph showing the ratio of the actual GOS formation rate over the theoretical GOS formation rate at 30° C. and 50° C. for the selected strains;

FIG. 4 is a graph showing the analysis of the difference in β-galactosidase activity from the initial screening experiments and the later GOS synthesis experiments in the selected strains; and

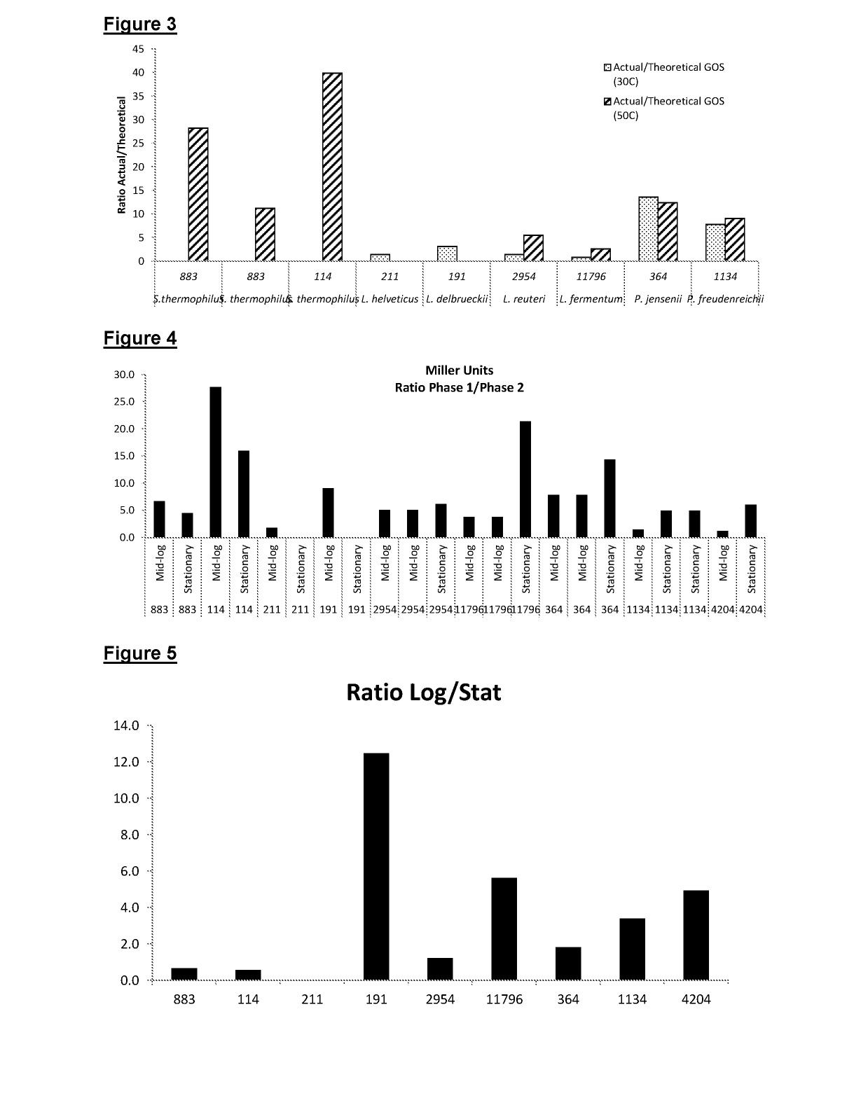

FIG. 5 shows the analysis (by means of the Log/Stat ratio) of the dependency of expression of β-galactosidase of the selected strains.

Mechanistically glycosidases are all transferases that use water as their preferred acceptor molecule. Under appropriate circumstance, however, such as high concentrations of substrate carbohydrate, these enzymes will transfer monosaccharide moieties from the substrate (acting as glycosyl donor) to other substrate or non-substrate carbohydrates (acting as glycosyl acceptor). Typically, the products of these reactions are complex mixtures containing all possible glycosidic linkages but in differing amounts. As the reactions are kinetically controlled, the linkage profile synthesised should map onto the rate constants for hydrolysis of those linkages by the producing enzyme. Consequently the oligosaccharides may be more readily metabolised by the producing organisms than by others in the gastrointestinal ecosystem. This approach has shown promise in laboratory testing.

It is possible, however in many enzyme synthesis reactions to include other carbohydrates which will act as acceptors in addition to the lactose. In this way, novel mixtures containing novel structures could be built up.

The basis of the present experiments was to reversibly use β-galactosidases in microorganisms so as to produce a novel GOS. Ordinarily, β-galactosidases would hydrolyse lactose. However, by changing the reaction conditions, in terms of substrate and temperature, the enzyme acts reversibly and generates an oligosaccharide version of the lactose (GOS).

### EXPERIMENTS

Experiments were conducted in two phases. The first phase screened 360 bacterial strains for the detection of β-galactosidase hydrolytic activity based on the breakdown of ortho-Nitrophenyl-β-galactoside (ONPG). The bacterial strains were selected from three bacterial genera (Streptococcus, Lactobacillus, Propionibacterium) and growth conditions were adjusted for each genus to attempt to improve the overall growth of each genus. β-galactosidase activity, expressed in Miller Units, was assessed and strains meeting the required activity were then put forward to the second phase. During the second phase, a feasibility study phase was conducted to screen the selected strains for their actual ability to synthesise GOS.

### Experiment 1

Screening of 360 Streptococcus, Lactobacillus and Propionibacterium strains was conducted for the detection of β-galactosidase hydrolytic activity based on the breakdown of ONPG. Growth conditions were adjusted for each genus and the total β-galactosidase activity assessed in miller units.

β-Galactosidase Activity in Streptococcus thermophilus

S. thermophilus strains were pre-grown from a −80° C. stock for 22 hours at 37° C. in 200 μl GM17 medium supplemented with 1% glucose in a standard 96 wells-plates. Cultures were re-diluted 100 fold to 1600 μl GM17 supplied with 1% glucose in deep-well plates. Growth was performed in anaerobic conditions at 37° C. for 22 hours. OD₆₀₀ was determined after a 10-fold dilution of the cultures. For the β-galactosidase activity, the cells were centrifuged at 5000×g at 4° C. and the pellets were subsequently lysed using 0.5 gram silicabeads (0.1 mm) in 800 μl 0.05M NaPi buffer pH=7.0. The supernatant was used for determining the β-galactosidase activity at 30° C. using a standard ONPG test protocol to assess the Miller Units. Table 1 below illustrates the results of those S. thermophilus strains which were screened using the above protocol.

TABLE 1

| | Total Bgal | | | | Bgal activity | |
|---|---|---|---|---|---|---|
| | activity | | Average OD600 | | (Miller Units) | |
| Strain no. | Average | Stdev | Average | Stdev | Average | Stdev |
| no. | μmol/min/l | | AU | | μmol/min/OD-Unit | |
| 883 | 2690 | 299 | 1.37 | 0.13 | 1960 | 83 |
| 885 | 1020 | 20 | 0.70 | 0.06 | 1462 | 103 |
| 882 | 906 | 21 | 0.68 | 0.05 | 1347 | 135 |
| 886 | 883 | 35 | 0.71 | 0.16 | 1291 | 238 |
| 884 | 506 | 90 | 0.40 | 0.10 | 1267 | 86 |
| 114 | 1565 | 81 | 1.37 | 0.22 | 1155 | 126 |
| 121 | 1459 | 118 | 1.29 | 0.19 | 1140 | 75 |
| 2310 | 1250 | 88 | 1.28 | 0.04 | 976 | 68 |
| 106 | 864 | 119 | 0.91 | 0.16 | 954 | 36 |
| 2106 | 869 | 30 | 1.11 | 0.26 | 815 | 196 |
| 105 | 195 | 44 | 0.27 | 0.02 | 718 | 101 |
| 2113 | 1267 | 116 | 1.84 | 0.01 | 690 | 61 |
| 109 | 803 | 43 | 1.18 | 0.20 | 690 | 83 |
| 130 | 1410 | 223 | 2.07 | 0.18 | 678 | 48 |
| 113 | 926 | 74 | 1.43 | 0.33 | 665 | 101 |
| 2320 | 885 | 29 | 1.36 | 0.10 | 653 | 50 |
| 1796 | 1123 | 47 | 1.79 | 0.20 | 633 | 77 |
| 110 | 1443 | 103 | 2.40 | 0.01 | 602 | 46 |
| 132 | 1263 | 451 | 2.34 | 0.57 | 600 | 338 |
| 2271 | 382 | 106 | 1.62 | 1.37 | 598 | 570 |
| 117 | 1139 | 76 | 1.94 | 0.21 | 597 | 100 |
| 2305 | 842 | 37 | 1.42 | 0.16 | 596 | 44 |
| 2306 | 711 | 44 | 1.20 | 0.00 | 591 | 39 |
| 2304 | 702 | 51 | 1.20 | 0.05 | 583 | 23 |
| 131 | 892 | 20 | 1.54 | 0.09 | 580 | 29 |
| 2308 | 910 | 69 | 1.68 | 0.20 | 545 | 33 |
| 2112 | 1086 | 67 | 2.05 | 0.13 | 530 | 17 |
| 116 | 966 | 91 | 1.91 | 0.46 | 519 | 82 |
| 2290 | 1289 | 58 | 2.51 | 0.08 | 513 | 12 |
| 2105 | 716 | 33 | 1.47 | 0.14 | 490 | 35 |
| 2279 | 1100 | 80 | 2.27 | 0.02 | 485 | 38 |
| 2291 | 462 | 239 | 1.11 | 0.78 | 479 | 120 |
| 2312 | 640 | 170 | 1.34 | 0.17 | 471 | 69 |
| 2107 | 637 | 74 | 1.37 | 0.22 | 469 | 35 |
| 1122 | 407 | 55 | 0.89 | 0.22 | 467 | 53 |
| 107 | 820 | 79 | 1.76 | 0.01 | 465 | 42 |
| 2318 | 457 | 43 | 1.02 | 0.12 | 448 | 14 |
| 1794 | 881 | 87 | 2.01 | 0.02 | 438 | 48 |
| 2315 | 553 | 137 | 1.31 | 0.27 | 419 | 20 |
| 108 | 528 | 150 | 1.33 | 0.58 | 418 | 69 |
| 111 | 718 | 165 | 1.75 | 0.13 | 417 | 124 |
| 2314 | 617 | 162 | 1.48 | 0.34 | 416 | 19 |
| 2311 | 587 | 76 | 1.41 | 0.09 | 416 | 28 |
| 2280 | 615 | 52 | 1.48 | 0.08 | 415 | 13 |
| 2289 | 688 | 27 | 1.76 | 0.02 | 391 | 12 |
| 2319 | 473 | 30 | 1.23 | 0.17 | 389 | 31 |
| 2086 | 542 | 158 | 1.45 | 0.52 | 382 | 30 |
| 2321 | 628 | 43 | 1.75 | 0.08 | 359 | 11 |
| 104 | 429 | 97 | 1.49 | 0.81 | 335 | 116 |
| 2313 | 754 | 47 | 2.29 | 0.26 | 332 | 27 |
| 2109 | 743 | 3 | 2.39 | 0.16 | 311 | 21 |
| 2307 | 437 | 59 | 1.44 | 0.04 | 305 | 50 |
| 1128 | 440 | 30 | 1.55 | 0.17 | 284 | 12 |
| 1953 | 564 | 39 | 2.03 | 0.19 | 281 | 46 |
| 2309 | 551 | 53 | 1.98 | 0.08 | 279 | 38 |
| 2316 | 605 | 48 | 2.31 | 0.08 | 262 | 12 |
| 122 | 406 | 85 | 1.56 | 0.02 | 260 | 57 |
| 2108 | 557 | 10 | 2.14 | 0.04 | 260 | 9 |
| 112 | 483 | 46 | 1.92 | 0.03 | 252 | 28 |
| 124 | 235 | 36 | 0.93 | 0.11 | 251 | 10 |
| 2288 | 678 | 16 | 2.78 | 0.17 | 244 | 21 |
| 2272 | 636 | 34 | 2.67 | 0.39 | 240 | 22 |
| 2317 | 498 | 117 | 2.15 | 0.06 | 233 | 61 |
| 1797 | 478 | 28 | 2.07 | 0.20 | 232 | 10 |
| 2285 | 430 | 37 | 1.89 | 0.00 | 228 | 19 |
| 126 | 322 | 30 | 1.42 | 0.06 | 227 | 12 |
| 119 | 288 | 11 | 1.32 | 0.05 | 219 | 16 |
| 2104 | 262 | 7 | 1.36 | 0.26 | 199 | 43 |
| 2269 | 260 | 13 | 1.35 | 0.04 | 192 | 15 |
| 127 | 274 | 10 | 1.57 | 0.06 | 175 | 1 |
| 2292 | 405 | 19 | 2.33 | 0.10 | 174 | 2 |
| 125 | 304 | 172 | 2.10 | 0.35 | 158 | 108 |
| 1951 | 342 | 51 | 2.30 | 0.15 | 148 | 13 |
| 2111 | 289 | 13 | 2.00 | 0.32 | 146 | 16 |
| 2278 | 313 | 4 | 2.16 | 0.04 | 145 | 4 |
| 2273 | 333 | 5 | 2.31 | 0.03 | 144 | 2 |
| 2277 | 313 | 16 | 2.20 | 0.11 | 143 | 1 |
| 2287 | 377 | 102 | 2.76 | 0.10 | 136 | 32 |
| 2286 | 301 | 10 | 2.34 | 0.10 | 129 | 10 |
| 2282 | 340 | 17 | 2.65 | 0.08 | 129 | 10 |
| 1950 | 332 | 9 | 2.63 | 0.12 | 127 | 9 |
| 123 | 211 | 23 | 1.92 | 0.03 | 110 | 14 |
| 118 | 256 | 16 | 2.37 | 0.08 | 108 | 3 |
| 2110 | 220 | 6 | 2.21 | 0.11 | 100 | 3 |
| 128 | 98 | 22 | 0.99 | 0.23 | 99.2 | 0.5 |
| 2283 | 204 | 20 | 2.33 | 0.04 | 87.8 | 10.0 |
| 2274 | 107 | 118 | 1.18 | 1.22 | 68.4 | 29.6 |
| 2284 | 142 | 27 | 2.40 | 0.12 | 59.0 | 8.2 |
| 2270 | 97 | 3 | 2.20 | 0.05 | 44.1 | 1.0 |
| 2275 | 44 | 46 | 0.06 | 0.09 | 29.7 | 1.5 |
| 133 | 5 | 2 | 1.45 | 0.08 | 3.7 | 1.4 |
| 129 | 4 | 1 | 3.01 | 0.21 | 1.4 | 0.5 |
| 2276 | 7 | 2 | 0.01 | 0.00 | 0.0 | 0.0 |
| 2281 | 5 | 3 | 0.00 | 0.02 | 0.0 | 0.0 |

### β-Galactosidase Activity in Propionibacterium

A range of Propionibacterium strains (including different species and sub-species) were pre-grown from a −80° C. stock for 72 hours at 30° C. in 200 μl LB medium supplemented with 1% glucose in a standard 96 well-plate. Cultures were re-diluted 100 fold to 1600 μl LB supplied with 1% glucose deep-well plates. Growth was performed in anaerobic conditions at 37° C. for 96 hours OD₆₀₀ was determined after a 10-fold dilution of the cultures. To assess β-galactosidase activity, cells were first centrifuged at 5000×g at 4° C. Then the pellets were lysed using 0.5 gram silicabeads (0.1 mm) in 800 μl 0.05M NaPi buffer pH=7.0. The supernatant was used for determining the β-galactosidase activity at 30° C. using a standard protocol.

Table 2 below illustrates the results of those Propionibacterium strains which were screened using the above protocol.

TABLE 2

| | | | | | Bgal activity | | | |
|---|---|---|---|---|---|---|---|---|
| | Total Bgal | | Average | | (Miller Units) | | | |
| Strain | activity | | OD600 | | Average | Stdev | | |
| no. | Average | Stdev | Average | Stdev | μmol/min/ | | | |
| no. | μmol/min/l | | AU | | OD-Unit | | Species | SubSpecies |
| 4204 | 17.6 | 3.9 | 2.94 | 0.10 | 6.0 | 1.5 | Propionibacterium | |
| | | | | | | | acidipropionici | |
| 380 | 15.8 | 0.7 | 2.86 | 0.34 | 5.6 | 0.4 | Propionibacterium sp. | |
| 2166 | 0.9 | 0.3 | 0.18 | 0.03 | 4.8 | 0.7 | Propionibacterium | freudenreichii |
| | | | | | | | freudenreichii | |
| 359 | 7.9 | 4.0 | 1.77 | 0.90 | 4.5 | 0.1 | Propionibacterium sp. | |
| 1134 | 10.6 | 1.0 | 2.49 | 0.07 | 4.2 | 0.3 | Propionibacterium | shermanii |
| | | | | | | | freudenreichii | |
| 364 | 10.8 | 1.3 | 2.91 | 0.03 | 3.7 | 0.4 | Propionibacterium | |
| | | | | | | | jensenii | |
| 2060 | 8.7 | 1.1 | 2.45 | 0.22 | 3.5 | 0.1 | Propionibacterium | shermanii |
| | | | | | | | freudenreichii | |
| 4199 | 5.9 | 1.4 | 1.69 | 0.44 | 3.5 | 0.1 | Propionibacterium | |
| | | | | | | | acidipropionici | |
| 4201 | 6.4 | 0.3 | 1.92 | 0.02 | 3.3 | 0.1 | Propionibacterium | |
| | | | | | | | acidipropionici | |
| 2175 | 6.8 | 0.2 | 2.04 | 0.06 | 3.3 | 0.2 | Propionibacterium | freudenreichii |
| | | | | | | | freudenreichii | |
| 2145 | 8.0 | 0.6 | 2.39 | 0.01 | 3.3 | 0.2 | Propionibacterium | freudenreichii |
| | | | | | | | freudenreichii | |
| 2168 | 7.3 | 1.2 | 2.36 | 0.07 | 3.1 | 0.6 | Propionibacterium | freudenreichii |
| | | | | | | | freudenreichii | |
| 2174 | 4.5 | 2.9 | 1.33 | 0.68 | 3.1 | 0.6 | Propionibacterium | freudenreichii |
| | | | | | | | freudenreichii | |
| 2173 | 6.4 | 0.4 | 2.06 | 0.08 | 3.1 | 0.3 | Propionibacterium | freudenreichii |
| | | | | | | | freudenreichii | |
| 384 | 1.5 | 0.3 | 0.53 | 0.11 | 2.9 | 1.1 | Propionibacterium | freudenreichii |
| | | | | | | | freudenreichii | |
| 2171 | 7.7 | 3.5 | 3.00 | 0.00 | 2.6 | 1.2 | Propionibacterium | freudenreichii |
| | | | | | | | freudenreichii | |
| 362 | 5.7 | 0.6 | 2.38 | 0.08 | 2.4 | 0.3 | Propionibacterium | |
| | | | | | | | acidipropionici | |
| 2172 | 4.4 | 2.2 | 1.83 | 0.34 | 2.3 | 0.8 | Propionibacterium | freudenreichii |
| | | | | | | | freudenreichii | |
| 360 | 0.6 | 0.2 | 0.14 | 0.22 | 2.2 | 0.3 | Propionibacterium | shermanii |
| | | | | | | | freudenreichii | |
| 2156 | 4.4 | 1.8 | 2.01 | 0.46 | 2.1 | 0.4 | Propionibacterium | freudenreichii |
| | | | | | | | freudenreichii | |
| 2541 | 3.2 | 2.7 | 1.65 | 0.18 | 2.1 | 1.9 | Propionibacterium sp. | |
| 2149 | 5.4 | 3.0 | 2.70 | 0.04 | 2.0 | 1.1 | Propionibacterium | freudenreichii |
| | | | | | | | freudenreichii | |
| 375 | 5.4 | 0.5 | 3.00 | 0.00 | 1.8 | 0.2 | Propionibacterium | |
| | | | | | | | acidipropionici | |
| 2169 | 4.7 | 0.6 | 2.68 | 0.05 | 1.8 | 0.2 | Propionibacterium | freudenreichii |
| | | | | | | | freudenreichii | |
| 2146 | 3.4 | 0.2 | 2.37 | 0.33 | 1.4 | 0.1 | Propionibacterium | freudenreichii |
| | | | | | | | freudenreichii | |
| 374 | 3.2 | 5.4 | 2.42 | 0.01 | 1.3 | 2.2 | Propionibacterium | shermanii |
| | | | | | | | freudenreichii | |
| 2167 | 2.9 | 0.6 | 2.23 | 0.06 | 1.3 | 0.2 | Propionibacterium | freudenreichii |
| | | | | | | | freudenreichii | |
| 2160 | 3.2 | 1.1 | 2.63 | 0.29 | 1.2 | 0.3 | Propionibacterium | freudenreichii |
| | | | | | | | freudenreichii | |
| 2150 | 0.9 | 0.5 | 1.24 | 1.04 | 1.1 | 0.7 | Propionibacterium | freudenreichii |
| | | | | | | | freudenreichii | |
| 2159 | 2.7 | 1.1 | 2.33 | 0.30 | 1.1 | 0.3 | Propionibacterium | freudenreichii |
| | | | | | | | freudenreichii | |
| 2543 | 2.1 | 0.3 | 1.88 | 0.01 | 1.1 | 0.2 | Propionibacterium sp. | |
| 371 | 1.2 | 0.0 | 1.10 | 0.13 | 1.1 | 0.1 | Propionibacterium | shermanii |
| | | | | | | | freudenreichii | |
| 4200 | 2.6 | 0.4 | 2.37 | 0.11 | 1.1 | 0.1 | Propionibacterium | |
| | | | | | | | acidipropionici | |
| 2178 | 1.2 | 0.2 | 1.33 | 0.54 | 1.1 | 0.6 | Propionibacterium | freudenreichii |
| | | | | | | | freudenreichii | |
| 2164 | 2.4 | 0.5 | 2.28 | 0.04 | 1.1 | 0.3 | Propionibacterium | freudenreichii |
| | | | | | | | freudenreichii | |
| 2162 | 2.4 | 1.9 | 2.04 | 1.23 | 1.0 | 0.3 | Propionibacterium | freudenreichii |
| | | | | | | | freudenreichii | |
| 367 | 2.0 | 0.5 | 1.93 | 0.08 | 1.0 | 0.2 | Propionibacterium | shermanii |
| | | | | | | | freudenreichii | |
| 2068 | 2.2 | 0.3 | 2.13 | 0.12 | 1.0 | 0.1 | Propionibacterium | shermanii |
| | | | | | | | freudenreichii | |
| 2177 | 1.9 | 0.3 | 1.86 | 0.12 | 1.0 | 0.1 | Propionibacterium | freudenreichii |
| | | | | | | | freudenreichii | |
| 2165 | 2.4 | 1.0 | 2.36 | 0.02 | 1.0 | 0.4 | Propionibacterium | freudenreichii |
| | | | | | | | freudenreichii | |
| 2155 | 2.7 | 0.1 | 2.65 | 0.11 | 1.0 | 0.1 | Propionibacterium | freudenreichii |
| | | | | | | | freudenreichii | |
| 2069 | 1.9 | 0.7 | 1.86 | 0.69 | 1.0 | 0.1 | Propionibacterium | shermanii |
| | | | | | | | freudenreichii | |
| 2161 | 2.2 | 1.3 | 2.17 | 0.31 | 1.0 | 0.5 | Propionibacterium | freudenreichii |
| | | | | | | | freudenreichii | |
| 2066 | 2.4 | 0.2 | 2.47 | 0.15 | 1.0 | 0.0 | Propionibacterium | freudenreichii |
| | | | | | | | freudenreichii | |
| 365 | 2.4 | 0.2 | 2.50 | 0.03 | 0.9 | 0.1 | Propionibacterium | shermanii |
| | | | | | | | freudenreichii | |
| 2544 | 2.2 | 0.4 | 2.32 | 0.02 | 0.9 | 0.2 | Propionibacterium sp. | |
| 2158 | 2.1 | 1.2 | 2.24 | 0.06 | 0.9 | 0.5 | Propionibacterium | freudenreichii |
| | | | | | | | freudenreichii | |
| 361 | 2.7 | 2.1 | 2.96 | 0.09 | 0.9 | 0.7 | Propionibacterium | shermanii |
| | | | | | | | thoenni | |
| 2163 | 1.9 | 0.9 | 2.11 | 0.20 | 0.9 | 0.3 | Propionibacterium | freudenreichii |
| | | | | | | | freudenreichii | |
| 2154 | 2.2 | 0.2 | 2.56 | 0.16 | 0.9 | 0.0 | Propionibacterium | freudenreichii |
| | | | | | | | freudenreichii | |
| 382 | 2.1 | 0.9 | 2.41 | 0.83 | 0.8 | 0.1 | Propionibacterium sp. | |
| 379 | 0.9 | 0.4 | 1.09 | 0.07 | 0.8 | 0.4 | Propionibacterium | freudenreichii |
| | | | | | | | freudenreichii | |
| 2542 | 1.9 | 1.2 | 2.32 | 0.05 | 0.8 | 0.5 | Propionibacterium sp. | |
| 372 | 1.6 | 0.4 | 2.25 | 0.04 | 0.7 | 0.2 | Propionibacterium | shermanii |
| | | | | | | | freudenreichii | |
| 2157 | 2.0 | 1.3 | 2.61 | 0.38 | 0.7 | 0.4 | Propionibacterium | freudenreichii |
| | | | | | | | freudenreichii | |
| 369 | 1.4 | 0.1 | 2.04 | 0.04 | 0.7 | 0.1 | Propionibacterium | shermanii |
| | | | | | | | freudenreichii | |
| 1256 | 1.7 | 0.9 | 2.40 | 0.29 | 0.7 | 0.3 | Propionibacterium sp. | |
| 2144 | 1.0 | 0.2 | 0.93 | 1.11 | 0.6 | 0.0 | Propionibacterium | freudenreichii |
| | | | | | | | freudenreichii | |
| 2179 | 1.3 | 0.3 | 2.12 | 0.03 | 0.6 | 0.1 | Propionibacterium | freudenreichii |
| | | | | | | | freudenreichii | |
| 2170 | 1.4 | 0.2 | 2.33 | 0.04 | 0.6 | 0.1 | Propionibacterium | freudenreichii |
| | | | | | | | freudenreichii | |
| 2336 | 1.4 | 0.8 | 2.48 | 0.05 | 0.6 | 0.3 | Propionibacterium | shermanii |
| | | | | | | | freudenreichii | |
| 2181 | 1.0 | 0.2 | 1.82 | 0.36 | 0.5 | 0.0 | Propionibacterium | freudenreichii |
| | | | | | | | freudenreichii | |
| 2176 | 1.6 | 0.1 | 3.13 | 0.15 | 0.5 | 0.0 | Propionibacterium | freudenreichii |
| | | | | | | | freudenreichii | |
| 2147 | 1.1 | 0.5 | 2.10 | 0.67 | 0.5 | 0.1 | Propionibacterium | freudenreichii |
| | | | | | | | freudenreichii | |
| 370 | 1.0 | 0.2 | 2.05 | 0.06 | 0.5 | 0.1 | Propionibacterium | shermanii |
| | | | | | | | freudenreichii | |
| 383 | 0.8 | 0.3 | 1.87 | 0.80 | 0.5 | 0.3 | Propionibacterium | freudenreichii |
| | | | | | | | freudenreichii | |
| 2663 | 1.2 | 0.7 | 2.60 | 0.04 | 0.5 | 0.3 | Propionibacterium | shermanii |
| | | | | | | | freudenreichii | |
| 2182 | 1.2 | 0.8 | 2.62 | 0.09 | 0.5 | 0.3 | Propionibacterium | freudenreichii |
| | | | | | | | freudenreichii | |
| 2151 | 1.0 | 0.1 | 2.28 | 0.26 | 0.4 | 0.1 | Propionibacterium | freudenreichii |
| | | | | | | | freudenreichii | |
| 2143 | 0.5 | 0.0 | 1.14 | 0.06 | 0.4 | 0.0 | Propionibacterium | freudenreichii |
| | | | | | | | freudenreichii | |
| 2152 | 0.9 | 0.1 | 2.45 | 0.19 | 0.4 | 0.1 | Propionibacterium | freudenreichii |
| | | | | | | | freudenreichii | |
| 2007 | 0.9 | 0.2 | 2.40 | 0.04 | 0.4 | 0.1 | Propionibacterium | shermanii |
| | | | | | | | freudenreichii | |
| 2065 | 0.9 | 0.1 | 2.39 | 0.11 | 0.4 | 0.0 | Propionibacterium | shermanii |
| | | | | | | | freudenreichii | |
| 363 | 0.6 | 0.1 | 1.66 | 0.07 | 0.3 | 0.1 | Propionibacterium | freudenreichii |
| | | | | | | | freudenreichii | |
| 2148 | 0.7 | 0.2 | 1.97 | 0.07 | 0.3 | 0.1 | Propionibacterium | freudenreichii |
| | | | | | | | freudenreichii | |
| 2180 | 1.0 | 0.1 | 3.00 | 0.00 | 0.3 | 0.0 | Propionibacterium | freudenreichii |
| | | | | | | | freudenreichii | |
| 2067 | 0.6 | 0.1 | 1.92 | 0.01 | 0.3 | 0.0 | Propionibacterium | shermanii |
| | | | | | | | freudenreichii | |
| 1219 | 0.4 | 0.2 | 2.27 | 0.06 | 0.2 | 0.1 | Propionibacterium | freudenreichii |
| | | | | | | | freudenreichii | |

### β-Galactosidase Activity in Lactobacillus

A range of Lactobacillus strains (including different species and sub-species) were pre-grown from a −80° C. stock for 48 hours at either 30° C. or 37° C. in 200 μl MRS medium in a standard 96 wells-plate in appropriate aerobiosis conditions. Cultures were re-diluted 100 fold to 1600 μl MRS medium in deep-well plates. Growth was performed in anaerobic conditions at 37° C. for 40 hours. OD₆₀₀ was determined after a 10-fold dilution of the cultures. For the β-galactosidase activity, cells were centrifuged at 5000×g at 4° C. The pellets were subsequently lysed using 0.5 gram silicabeads (0.1 mm) in 800 μl 0.05M NaPi buffer pH=7.0. The supernatant was then used for determining the β-galactosidase activity at 30° C. using a standard protocol.

Table 3 below illustrates the results of those Lactobacillus strains which were screened using the above protocol.

TABLE 3

| | | | | | Bgal activity | | | |
|---|---|---|---|---|---|---|---|---|
| | Total Bgal | | Average | | (Miller Units) | | | |
| Strain | activity | | OD600 | | Average | Stdev | | |
| no. | Average | Stdev | Average | Stdev | μmol/min/ | | | |
| no. | μmol/min/l | | AU | | OD-Unit | | Species | Subspecies |
| 194 | 874 | 19 | 0.86 | 0.03 | 1019 | 59 | Lactobacillus | bulgaricus |
| | | | | | | | delbrueckii | |
| 191 | 1775 | 111 | 1.89 | 0.01 | 937 | 62 | Lactobacillus | bulgaricus |
| | | | | | | | delbrueckii | |
| 203 | 1525 | 96 | 1.90 | 0.16 | 804 | 16 | Lactobacillus | bulgaricus |
| | | | | | | | delbrueckii | |
| 192 | 1171 | 320 | 2.03 | 0.53 | 620 | 320 | Lactobacillus | bulgaricus |
| | | | | | | | delbrueckii | |
| 195 | 1157 | 256 | 2.14 | 0.02 | 541 | 114 | Lactobacillus | bulgaricus |
| | | | | | | | delbrueckii | |
| 202 | 1127 | 211 | 2.08 | 0.10 | 540 | 76 | Lactobacillus | bulgaricus |
| | | | | | | | delbrueckii | |
| 187 | 1084 | 197 | 2.51 | 0.79 | 442 | 61 | Lactobacillus | bulgaricus |
| | | | | | | | delbrueckii | |
| 189 | 1343 | 201 | 3.25 | 0.08 | 413 | 52 | Lactobacillus | bulgaricus |
| | | | | | | | delbrueckii | |
| 211 | 1205 | 37 | 3.06 | 0.48 | 398 | 51 | Lactobacillus | |
| | | | | | | | helveticus | |
| 1456 | 1514 | 23 | 3.95 | 0.12 | 384 | 6 | Lactobacillus | |
| | | | | | | | crispatus | |
| 204 | 1203 | 102 | 3.81 | 0.08 | 315 | 20 | Lactobacillus | bulgaricus |
| | | | | | | | delbrueckii | |
| 186 | 550 | 196 | 1.92 | 0.28 | 282 | 62 | Lactobacillus | bulgaricus |
| | | | | | | | delbrueckii | |
| 3273 | 866 | 7 | 3.40 | 0.17 | 255 | 14 | Lactobacillus | |
| | | | | | | | helveticus | |
| 1795 | 391 | 185 | 1.79 | 0.38 | 213 | 58 | Lactobacillus | |
| | | | | | | | delbrueckii | |
| 190 | 18 | 11 | 0.13 | 0.06 | 150 | 0 | Lactobacillus | bulgaricus |
| | | | | | | | delbrueckii | |
| *301 | 500 | 37 | 3.48 | 0.04 | 144 | 12 | Lactobacillus | |
| | | | | | | | buchneri | |
| 1572 | 207 | 254 | 1.38 | 1.97 | 139 | | Lactobacillus | |
| | | | | | | | fermentum | |
| 2954 | 286 | 2 | 2.58 | 0.02 | 111 | 0 | Lactobacillus | |
| | | | | | | | reuteri | |
| 3416 | 261 | 0 | 2.49 | 0.11 | 105 | 5 | Lactobacillus | |
| | | | | | | | reuteri | |
| 226 | 304 | 2 | 3.04 | 0.22 | 101 | 7 | Lactobacillus | |
| | | | | | | | acidophilus | |
| 3909 | 310 | 6 | 3.29 | 0.11 | 94.0 | 1.2 | Lactobacillus | |
| | | | | | | | reuteri | |
| LR92 | 276 | 3 | 3.04 | 0.25 | 90.9 | 6.3 | L. reuteri | |
| 2955 | 244 | 7 | 2.72 | 0.02 | 89.7 | 1.9 | Lactobacillus | |
| | | | | | | | reuteri | |
| LR1 | 306 | 5 | 3.68 | 0.70 | 84.5 | 14.5 | L. rhamsnosus | |
| 2955 | 235 | 25 | 2.82 | 0.13 | 83.8 | 12.6 | Lactobacillus | |
| | | | | | | | reuteri | |
| 3423 | 246 | 28 | 3.02 | 0.04 | 81.3 | 8.1 | Lactobacillus | |
| | | | | | | | reuteri | |
| 3423 | 220 | 1 | 2.90 | 0.02 | 75.7 | 0.3 | Lactobacillus | |
| | | | | | | | reuteri | |
| 3422 | 196 | 4 | 2.69 | 0.37 | 73.5 | 8.6 | Lactobacillus | |
| | | | | | | | reuteri | |
| LA1 | 294 | 12 | 4.07 | 0.18 | 72.3 | 6.2 | L. acidophilus | |
| 11796 | 190 | 22 | 2.74 | 0.00 | 69.2 | 8.1 | Lactobacillus | |
| | | | | | | | fermentum | |
| 634 | 183 | 10 | 2.96 | 0.16 | 61.9 | 0.1 | Lactobacillus | |
| | | | | | | | acidophilus | |
| 3797 | 159 | 18 | 2.66 | 0.04 | 59.8 | 6.0 | Lactobacillus | |
| | | | | | | | gasseri | |
| 227 | 14 | 14 | 0.26 | 0.29 | 52.0 | 0.0 | Lactobacillus | |
| | | | | | | | acidophilus | |
| 3421 | 152 | 11 | 3.01 | 0.18 | 50.6 | 0.4 | Lactobacillus | |
| | | | | | | | reuteri | |
| 3418 | 169 | 8 | 3.46 | 0.19 | 49.1 | 5.0 | Lactobacillus | |
| | | | | | | | reuteri | |
| 197 | 59 | 1 | 1.25 | 0.12 | 47.2 | 3.6 | Lactobacillus | bulgaricus |
| | | | | | | | delbrueckii | |
| D | 119 | 3 | 2.86 | 0.15 | 41.5 | 1.1 | Lactobacillus | |
| 3106 | 141 | 184 | 3.31 | 0.13 | 41.4 | 53.8 | Lactobacillus | |
| | | | | | | | acidophilus | |
| 692 | 73 | 14 | 1.86 | 0.26 | 39.0 | 1.9 | Lactobacillus | |
| | | | | | | | acidophilus | |
| 3117 | 131 | 180 | 2.26 | 1.73 | 38.6 | 50.0 | Lactobacillus | |
| | | | | | | | amylolyticus | |
| 881 | 154 | 91 | 4.30 | 0.02 | 35.7 | 21.0 | Lactobacillus | |
| | | | | | | | salivarius | |
| *298 | 103 | 1 | 2.99 | 0.06 | 34.6 | 1.0 | Lactobacillus | |
| | | | | | | | buchneri | |
| 205 | 33 | 15 | 1.04 | 0.45 | 31.7 | 1.0 | Lactobacillus | bulgaricus |
| | | | | | | | delbrueckii | |
| 1178 | 7 | 6 | 0.17 | 0.25 | 30.9 | 0.0 | Lactobacillus | |
| | | | | | | | crispatus | |
| 3419 | 90 | 15 | 2.92 | 0.09 | 30.8 | 4.1 | Lactobacillus | |
| | | | | | | | reuteri | |
| 3419 | 91 | 6 | 3.08 | 0.01 | 29.6 | 1.8 | Lactobacillus | |
| | | | | | | | reuteri | |
| 2487 | 82 | 6 | 2.91 | 0.30 | 28.4 | 0.7 | Lactobacillus | |
| | | | | | | | brevis | |
| 881 | 105 | 126 | 4.28 | 0.80 | 27.8 | 34.6 | Lactobacillus | |
| | | | | | | | salivarius | |
| 2478 | 84 | 17 | 3.04 | 0.08 | 27.6 | 6.2 | Lactobacillus | |
| | | | | | | | brevis | |
| 1178 | 19 | 16 | 0.85 | 0.82 | 25.8 | 5.9 | Lactobacillus | |
| | | | | | | | crispatus | |
| 1688 | 75 | 4 | 2.92 | 0.17 | 25.8 | 3.0 | Lactobacillus | |
| | | | | | | | fermentum | |
| 1177 | 60 | 7 | 2.42 | 0.36 | 25.0 | 0.8 | Lactobacillus | |
| | | | | | | | helveticus | |
| 2472 | 73 | 2 | 3.08 | 0.06 | 23.7 | 0.3 | Lactobacillus | |
| | | | | | | | brevis | |
| 1533 | 77 | 97 | 2.81 | 0.81 | 23.4 | 27.6 | Lactobacillus | |
| | | | | | | | reuteri | |
| 2481 | 70 | 2 | 3.11 | 0.14 | 22.6 | 0.3 | Lactobacillus | |
| | | | | | | | brevis | |
| 1229 | 89 | 123 | 4.72 | 0.13 | 18.4 | 25.6 | Lactobacillus | |
| | | | | | | | jensenii | |
| *297 | 50 | 5 | 2.78 | 0.02 | 17.9 | 1.6 | Lactobacillus | |
| | | | | | | | buchneri | |
| 3417 | 38 | 2 | 2.16 | 0.04 | 17.8 | 1.2 | Lactobacillus | |
| | | | | | | | reuteri | |
| 198 | 23 | 4 | 1.34 | 0.04 | 17.4 | 3.5 | Lactobacillus | bulgaricus |
| | | | | | | | delbrueckii | |
| 3420 | 53 | 5 | 3.14 | 0.24 | 17.0 | 0.3 | Lactobacillus | |
| | | | | | | | reuteri | |
| 3473 | 54 | 53 | 3.11 | 0.36 | 16.4 | 15.0 | Lactobacillus | |
| | | | | | | | helveticus | |
| 207 | 3 | 1 | 0.14 | 0.15 | 16.3 | 0.0 | Lactobacillus | |
| | | | | | | | helveticus | |
| *302 | 33 | 2 | 2.05 | 0.02 | 16.0 | 0.9 | Lactobacillus | |
| | | | | | | | buchneri | |
| 3222 | 27 | 14 | 1.67 | 0.20 | 15.9 | 6.5 | Lactobacillus | |
| | | | | | | | fermentum | |
| 2480 | 43 | 55 | 3.89 | 1.56 | 15.2 | 20.2 | Lactobacillus | bulgaricus |
| | | | | | | | brevis | |
| 196 | 41 | 3 | 2.73 | 0.09 | 15.0 | 0.6 | Lactobacillus | bulgaricus |
| | | | | | | | delbrueckii | |
| 3329 | 13 | 1 | 1.00 | 0.23 | 13.2 | 4.4 | Lactobacillus | |
| | | | | | | | panis | |
| 695 | 45 | 16 | 3.55 | 0.09 | 12.8 | 4.7 | Lactobacillus | |
| | | | | | | | crispatus | |
| 1457 | 44 | 6 | 3.90 | 0.73 | 11.4 | 0.7 | Lactobacillus | |
| | | | | | | | crispatus | |
| 695 | 35 | 3 | 3.22 | 0.07 | 10.7 | 0.6 | Lactobacillus | |
| | | | | | | | crispatus | |
| 30226 | 34 | 2 | 3.20 | 0.02 | 10.6 | 0.8 | Lactobacillus | |
| | | | | | | | fermentum | |
| 1161 | 6 | 1 | 0.35 | 0.30 | 10.5 | 0.2 | Lactobacillus | |
| | | | | | | | pentosus | |
| 216 | 31 | 5 | 2.94 | 0.40 | 10.4 | 0.4 | Lactobacillus | |
| | | | | | | | helveticus | |
| 198 | 16 | 18 | 1.52 | 0.17 | 10.2 | 10.5 | Lactobacillus | bulgaricus |
| | | | | | | | delbrueckii | |
| 619 | 3 | 0 | 0.40 | 0.01 | 8.6 | 0.5 | Lactobacillus | |
| | | | | | | | helveticus | |
| *300 | 23 | 3 | 2.83 | 0.18 | 8.3 | 0.4 | Lactobacillus | |
| | | | | | | | buchneri | |
| 212 | 26 | 0 | 3.46 | 0.46 | 7.7 | 1.0 | Lactobacillus | |
| | | | | | | | helveticus | |
| 307 | 20 | 2 | 2.76 | 0.05 | 7.4 | 0.7 | Lactobacillus | |
| | | | | | | | fermentum | |
| 1519 | 4 | 1 | 0.33 | 0.31 | 7.3 | 0.1 | Lactobacillus | |
| | | | | | | | diolivorans | |
| 618 | 23 | 7 | 3.22 | 0.18 | 6.9 | 1.9 | Lactobacillus | |
| | | | | | | | helveticus | |
| 3427 | 30 | 1 | 4.49 | 0.64 | 6.7 | 0.8 | Lactobacillus | |
| | | | | | | | rhamnosus | |
| 285 | 2 | 1 | 0.25 | 0.18 | 6.5 | 1.0 | Lactobacillus | |
| | | | | | | | pentosus | |
| 225 | 16 | 1 | 2.58 | 0.15 | 6.4 | 0.0 | Lactobacillus | |
| | | | | | | | acidophilus | |
| 233 | 15 | 1 | 2.39 | 0.01 | 6.1 | 0.3 | Lactobacillus | |
| | | | | | | | acidophilus | |
| 1518 | 3 | 1 | 0.35 | 0.29 | 5.9 | 0.1 | Lactobacillus | |
| | | | | | | | diolivorans | |
| 206 | 15 | 3 | 2.66 | 0.49 | 5.8 | 0.1 | Lactobacillus | |
| | | | | | | | helveticus | |
| 1307 | 3 | 0 | 0.28 | 0.31 | 5.8 | 0.0 | Lactobacillus | |
| | | | | | | | buchneri | |
| 3191 | 20 | 19 | 3.46 | 0.31 | 5.7 | 5.1 | Lactobacillus | |
| | | | | | | | crispatus | |
| 3098 | 14 | 0 | 2.60 | 0.15 | 5.3 | 0.3 | Lactobacillus | |
| | | | | | | | acidophilus | |
| 223 | 12 | 0 | 2.31 | 0.14 | 5.2 | 0.2 | Lactobacillus | |
| | | | | | | | acidophilus | |
| 3212 | 13 | 0 | 2.68 | 0.36 | 5.1 | 0.8 | Lactobacillus | |
| | | | | | | | delbrueckii | |
| 199 | 8 | 1 | 1.77 | 0.16 | 4.4 | 0.9 | Lactobacillus | bulgaricus |
| | | | | | | | delbrueckii | |
| 267 | 10 | 0 | 2.77 | 0.30 | 3.7 | 0.5 | Lactobacillus | |
| | | | | | | | acidophilus | |
| 294 | 10 | 1 | 2.91 | 0.01 | 3.5 | 0.2 | Lactobacillus | |
| | | | | | | | fermentum | |
| 266 | 9 | 0 | 2.84 | 0.04 | 3.2 | 0.0 | Lactobacillus | |
| | | | | | | | acidophilus | |
| 3118 | 3 | 1 | 0.93 | 0.03 | 3.0 | 0.7 | Lactobacillus | |
| | | | | | | | amylolyticus | |
| 3119 | 3 | 0 | 1.10 | 0.08 | 2.9 | 0.5 | Lactobacillus | |
| | | | | | | | amylolyticus | |
| 3114 | 4 | 0 | 1.23 | 0.15 | 2.9 | 0.3 | Lactobacillus | |
| | | | | | | | amylolyticus | |
| 3115 | 4 | 1 | 1.35 | 0.03 | 2.8 | 0.6 | Lactobacillus | |
| | | | | | | | amylolyticus | |
| 193 | 6 | 1 | 2.11 | 0.14 | 2.7 | 0.5 | Lactobacillus | lactis |
| | | | | | | | delbrueckii | |
| 3208 | 2 | 1 | 0.91 | 0.03 | 2.6 | 1.1 | Lactobacillus | |
| | | | | | | | delbrueckii | |
| 241 | 3 | 0 | 1.13 | 0.01 | 2.2 | 0.2 | Lactobacillus | |
| | | | | | | | casei | |
| 3116 | 4 | 1 | 1.72 | 0.01 | 2.2 | 0.6 | Lactobacillus | |
| | | | | | | | amylolyticus | |
| 3234 | 7 | 0 | 3.27 | 0.25 | 2.1 | 0.2 | Lactobacillus | |
| | | | | | | | fermentum | |
| 3117 | 2 | 1 | 1.00 | 0.00 | 2.0 | 0.7 | Lactobacillus | |
| | | | | | | | amylolyticus | |
| 3122 | 3 | 1 | 1.81 | 0.03 | 1.9 | 0.6 | Lactobacillus | |
| | | | | | | | amylolyticus | |
| 222 | 4 | 0 | 2.46 | 0.23 | 1.7 | 0.2 | Lactobacillus | |
| | | | | | | | helveticus | |
| 871 | 3 | 1 | 1.82 | 0.09 | 1.7 | 0.3 | Lactobacillus | |
| | | | | | | | crispatus | |
| *3130 | 1 | 0 | 0.60 | 0.14 | 1.6 | 0.3 | Lactobacillus | |
| | | | | | | | amylovorus | |
| 1356 | 5 | 0 | 3.03 | 0.05 | 1.5 | 0.1 | Lactobacillus | |
| | | | | | | | graminus | |
| 3121 | 3 | 1 | 1.96 | 0.08 | 1.5 | 0.2 | Lactobacillus | |
| | | | | | | | amylolyticus | |
| C | 4 | 1 | 2.58 | 0.12 | 1.4 | 0.5 | Lactobacillus | |
| 3123 | 2 | 0 | 1.40 | 0.04 | 1.4 | 0.3 | Lactobacillus | |
| | | | | | | | amylolyticus | |
| 3120 | 2 | 0 | 1.36 | 0.03 | 1.3 | 0.4 | Lactobacillus | |
| | | | | | | | amylolyticus | |
| 3301 | 3 | 0 | 2.75 | 0.14 | 1.3 | 0.0 | Lactobacillus | |
| | | | | | | | johnsonii | |
| 3299 | 3 | 1 | 2.47 | 0.11 | 1.1 | 0.5 | Lactobacillus | |
| | | | | | | | johnsonii | |
| *3245 | 1 | 0 | 0.61 | 0.26 | 1.1 | 0.1 | Lactobacillus | |
| | | | | | | | gasseri | |
| *3128 | 4 | 0 | 3.76 | 0.15 | 1.1 | 0.1 | Lactobacillus | |
| | | | | | | | amylovorus | |
| 229 | 3 | 1 | 2.68 | 0.09 | 1.1 | 0.3 | Lactobacillus | |
| | | | | | | | acidophilus | |
| 2828 | 3 | 1 | 3.06 | 0.19 | 1.0 | 0.3 | Lactobacillus | |
| | | | | | | | plantarum | |
| 3114 | 2 | 1 | 2.53 | 1.62 | 1.0 | 0.9 | Lactobacillus | |
| | | | | | | | amylolyticus | |
| 240 | 4 | 2 | 3.80 | 0.11 | 0.9 | 0.6 | Lactobacillus | |
| | | | | | | | casei | |
| 3211 | 2 | 1 | 1.47 | 2.08 | 0.9 | 0.0 | Lactobacillus | |
| | | | | | | | delbrueckii | |
| 3300 | 4 | 0 | 4.32 | 1.12 | 0.9 | 0.2 | Lactobacillus | |
| | | | | | | | johnsonii | |
| 224 | 2 | 0 | 2.09 | 0.16 | 0.8 | 0.1 | Lactobacillus | |
| | | | | | | | acidophilus | |
| 3431 | 1 | 0 | 1.01 | 0.07 | 0.8 | 0.0 | Lactobacillus | |
| | | | | | | | rhamnosus | |
| 645 | 3 | 0 | 3.73 | 0.09 | 0.8 | 0.1 | Lactobacillus | |
| | | | | | | | acidophilus | |
| *3251 | 1 | 0 | 0.91 | 0.25 | 0.7 | 0.1 | Lactobacillus | |
| | | | | | | | gasseri | |
| 265 | 4 | 3 | 5.10 | 0.00 | 0.7 | 0.5 | Lactobacillus | |
| | | | | | | | crispatus | |
| 242 | 2 | 0 | 3.76 | 0.13 | 0.7 | 0.0 | Lactobacillus | |
| | | | | | | | casei | |
| 3436 | 2 | 0 | 3.02 | 0.02 | 0.6 | 0.0 | Lactobacillus | |
| | | | | | | | rhamnosus | |
| 2830 | 3 | 0 | 3.95 | 0.07 | 0.6 | 0.0 | Lactobacillus | |
| | | | | | | | plantarum | |
| 1479 | 3 | 0 | 4.12 | 0.16 | 0.6 | 0.1 | Lactobacillus | paracasei |
| | | | | | | | paracasei | |
| 2691 | 1 | 0 | 2.51 | 0.30 | 0.5 | 0.1 | Lactobacillus | |
| | | | | | | | plantarum | |
| 239 | 2 | 1 | 3.75 | 0.21 | 0.5 | 0.2 | Lactobacillus | |
| | | | | | | | casei | |
| 645 | 2 | 1 | 4.04 | 0.17 | 0.5 | 0.3 | Lactobacillus | |
| | | | | | | | acidophilus | |
| 880 | 2 | 1 | 3.67 | 0.13 | 0.5 | 0.2 | Lactobacillus | |
| | | | | | | | salivarius | |
| 238 | 1 | 0 | 3.16 | 0.14 | 0.5 | 0.1 | Lactobacillus | |
| | | | | | | | acidophilus | |
| 3350 | 2 | 1 | 3.52 | 0.21 | 0.4 | 0.2 | Lactobacillus | |
| | | | | | | | paracasei | |
| 2523 | 2 | 1 | 3.57 | 0.21 | 0.4 | 0.3 | Lactobacillus | |
| | | | | | | | helveticus | |
| 646 | 1 | 0 | 2.91 | 0.08 | 0.4 | 0.2 | Lactobacillus | |
| | | | | | | | acidophilus | |
| 3440 | 1 | 0 | 3.34 | 0.92 | 0.4 | 0.2 | Lactobacillus | |
| | | | | | | | rhamnosus | |
| 3429 | 1 | 0 | 3.70 | 0.05 | 0.4 | 0.0 | Lactobacillus | |
| | | | | | | | rhamnosus | |
| B | 1 | 1 | 3.23 | 0.03 | 0.4 | 0.2 | Lactobacillus | |
| 3428 | 2 | 0 | 4.34 | 0.37 | 0.4 | 0.1 | Lactobacillus | |
| | | | | | | | rhamnosus | |
| 259 | 1 | 0 | 1.31 | 1.84 | 0.4 | 0.0 | Lactobacillus | |
| | | | | | | | acidophilus | |
| *870 | 1 | 0 | 2.40 | 0.01 | 0.4 | 0.2 | Lactobacillus | |
| | | | | | | | amylovorus | |
| 3444 | 1 | 0 | 3.59 | 0.22 | 0.4 | 0.2 | Lactobacillus | |
| | | | | | | | rhamnosus | |
| 3302 | 1 | 0 | 2.03 | 0.06 | 0.4 | 0.0 | Lactobacillus | |
| | | | | | | | johnsonii | |
| 1353 | 1 | 0 | 2.89 | 0.02 | 0.4 | 0.1 | Lactobacillus | paracasei |
| | | | | | | | paracasei | |
| 101/37 | 2 | 1 | 5.15 | 0.04 | 0.3 | 0.2 | L. paracasei | |
| 3445 | 1 | 0 | 3.79 | 0.37 | 0.3 | 0.1 | Lactobacillus | |
| | | | | | | | rhamnosus | |
| 3443 | 2 | 0 | 4.65 | 0.30 | 0.3 | 0.0 | Lactobacillus | |
| | | | | | | | rhamnosus | |
| \hat{ }14D | 2 | 0 | 4.88 | 0.33 | 0.3 | 0.0 | L. plantarum | |
| 2937 | 1 | 0 | 4.50 | 0.08 | 0.3 | 0.0 | Lactobacillus | |
| | | | | | | | rhamnosus | |
| 3439 | 1 | 0 | 3.86 | 0.15 | 0.3 | 0.1 | Lactobacillus | |
| | | | | | | | rhamnosus | |
| 3434 | 1 | 0 | 4.39 | 0.26 | 0.3 | 0.1 | Lactobacillus | |
| | | | | | | | rhamnosus | |
| 3426 | 1 | 0 | 4.67 | 0.53 | 0.3 | 0.1 | Lactobacillus | |
| | | | | | | | rhamnosus | |
| 3303 | 1 | 0 | 2.42 | 0.08 | 0.3 | 0.1 | Lactobacillus | |
| | | | | | | | johnsonii | |
| *3246 | 1 | 0 | 2.46 | 0.05 | 0.3 | 0.1 | Lactobacillus | |
| | | | | | | | gasseri | |
| *1356 | 0 | 0 | 1.70 | 0.06 | 0.3 | 0.1 | Lactobacillus | |
| | | | | | | | graminus | |
| 3438 | 1 | 0 | 4.13 | 0.06 | 0.3 | 0.0 | Lactobacillus | |
| | | | | | | | rhamnosus | |
| 3442 | 1 | 0 | 5.01 | 0.34 | 0.3 | 0.1 | Lactobacillus | |
| | | | | | | | rhamnosus | |
| 3430 | 1 | 0 | 4.65 | 0.05 | 0.3 | 0.0 | Lactobacillus | |
| | | | | | | | rhamnosus | |
| *3201 | 1 | 0 | 3.33 | 0.23 | 0.3 | 0.1 | Lactobacillus | |
| | | | | | | | curvatus | |
| 3425 | 1 | 0 | 4.71 | 0.37 | 0.3 | 0.0 | Lactobacillus | |
| | | | | | | | rhamnosus | |
| 1226 | 1 | 0 | 4.65 | 0.13 | 0.3 | 0.1 | Lactobacillus | paracasei |
| | | | | | | | paracasei | |
| *3200 | 1 | 0 | 3.26 | 0.24 | 0.2 | 0.1 | Lactobacillus | |
| | | | | | | | curvatus | |
| 3433 | 1 | 0 | 3.04 | 0.09 | 0.2 | 0.1 | Lactobacillus | |
| | | | | | | | rhamnosus | |
| 3435 | 1 | 0 | 4.91 | 0.03 | 0.2 | 0.1 | Lactobacillus | |
| | | | | | | | rhamnosus | |
| E | 1 | 1 | 4.39 | 0.30 | 0.2 | 0.2 | Lactobacillus | |
| 2518 | 1 | 0 | 4.92 | 0.38 | 0.2 | 0.0 | Lactobacillus | paracasei |
| | | | | | | | paracasei | |
| *3249 | 0 | 0 | 1.72 | 0.03 | 0.2 | 0.0 | Lactobacillus | |
| | | | | | | | gasseri | |
| 636 | 1 | 0 | 4.34 | 0.05 | 0.2 | 0.0 | Lactobacillus | |
| | | | | | | | salivarius | |
| 638 | 1 | 1 | 5.64 | 0.02 | 0.2 | 0.1 | Lactobacillus | |
| | | | | | | | salivarius | |
| 3437 | 1 | 0 | 4.11 | 0.13 | 0.2 | 0.0 | Lactobacillus | |
| | | | | | | | rhamnosus | |
| A | 1 | 1 | 5.08 | 0.12 | 0.2 | 0.1 | Lactobacillus | |
| *872 | 1 | 0 | 2.90 | 0.21 | 0.2 | 0.0 | Lactobacillus | |
| | | | | | | | gasseri | |
| *3196 | 1 | 0 | 3.32 | 0.07 | 0.2 | 0.0 | Lactobacillus | |
| | | | | | | | curvatus | |
| *1357 | 1 | 0 | 3.19 | 0.01 | 0.2 | 0.1 | Lactobacillus | |
| | | | | | | | paralimentarius | |
| 3441 | 1 | 1 | 5.38 | 0.03 | 0.2 | 0.1 | Lactobacillus | |
| | | | | | | | rhamnosus | |
| *3202 | 1 | 0 | 3.25 | 0.12 | 0.2 | 0.1 | Lactobacillus | |
| | | | | | | | curvatus | |
| *3203 | 1 | 0 | 3.65 | 0.23 | 0.2 | 0.0 | Lactobacillus | |
| | | | | | | | curvatus | |
| 3432 | 1 | 0 | 5.00 | 0.28 | 0.2 | 0.0 | Lactobacillus | |
| | | | | | | | rhamnosus | |
| *3195 | 1 | 0 | 3.75 | 0.07 | 0.2 | 0.0 | Lactobacillus | |
| | | | | | | | curvatus | |
| *1228 | 1 | 0 | 3.69 | 0.08 | 0.1 | 0.0 | Lactobacillus | |
| | | | | | | | gasseri | |
| 3424 | 2 | 0 | 22.40 | 2.22 | 0.1 | 0.0 | Lactobacillus | |
| | | | | | | | rhamnosus | |
| 1531 | 1 | 0 | 0.01 | 0.01 | 0.0 | 0.0 | Lactobacillus | |
| | | | | | | | panis | |
| *3129 | 1 | 0 | −0.01 | 0.01 | 0.0 | 0.0 | Lactobacillus | |
| | | | | | | | amylovorus | |
| *3247 | 1 | 0 | 0.03 | 0.03 | 0.0 | 0.0 | Lactobacillus | |
| | | | | | | | gasseri | |
| *3248 | 0 | 0 | 0.07 | 0.02 | 0.0 | 0.0 | Lactobacillus | |
| | | | | | | | gasseri | |
| *3250 | 0 | 0 | 0.03 | 0.04 | 0.0 | 0.0 | Lactobacillus | |
| | | | | | | | gasseri | |
| *3252 | 1 | 0 | 0.65 | 0.93 | 0.0 | 0.0 | Lactobacillus | |
| | | | | | | | gasseri | |
| *3253 | 1 | 0 | −0.01 | 0.01 | 0.0 | 0.0 | Lactobacillus | |
| | | | | | | | gasseri | |
| *3254 | 1 | 0 | −0.01 | 0.00 | 0.0 | 0.0 | Lactobacillus | |
| | | | | | | | gasseri | |

(Note: All strains were grown at 37° C. in anaerobic conditions, except those strains denoted “*” which were grown at 30° C. in aerobic conditions or “\hat{ }” which were grown at 37° C. in aerobic conditions).

Strains having β-galactosidase activity value of greater than 60 Miller Units were identified and put forward for further assessment for potential GOS synthesis and initial optimisation studies. Two S. thermophilus strains were selected, 4 lactobacilli (L. helveticus, L. reuters, L. delbrueckii, L. fermentum) with miller unit output above 60 and one Lactobacillus (L. plantarum 2830) with β-galactosidase activity bellow 60 miller units (for a control) were analysed. As none of the Propionibacterium screened gave β-galactosidase levels above 60, those with the highest β-galactosidase levels producers of each species was also included in the next phase of the study.

### Analysis of GOS Production in the Chosen Strains

The following growth protocols were used for each species:

S. thermophilus—S. thermophilus strains were pre-grown from the −80° C. stock for 22 hours at 37° C. in 100 ml GM17 medium supplemented with 1% glucose in a closed 100 ml bottle. Cultures were then diluted 50, 200, 1000 and 4000-fold in a 1 litre bottle filled with GM17 medium supplemented with 1% glucose. Growth was performed at 37° C. for a set time that had been calculated to ensure a logarithmic culture and a stationary phase culture at the aimed time of harvesting.

Propionibacteria—Propionibacterium strains were pre-grown from the −80° C. stock for 72 hours at 30° C. in 100 ml LB medium supplied with 1% glucose. Cultures were diluted 50, 200, 1000 and 4000-fold in a 1 litre bottle filled with LB medium supplied with 1% glucose. Growth was performed at 30° C. for a set calculated time that had been calculated to ensure a logarithmic culture and a stationary phase culture at the aimed time of harvesting.

Lactobacilli—Lactobacillus strains were pre-grown from the −80° C. stock for 48 hours at 37° C. in 100 ml MRS medium. Cultures were diluted 50, 200, 1000 and 4000-fold in a 1 litre bottle filled with MRS medium supplemented with 1% glucose. Growth was performed at 37° C. for a set calculated time that had been calculated to ensure a logarithmic culture and a stationary phase culture at the aimed time of harvesting.

### Analysis of β-Galactosidase Activity in the Chosen Strains

To analyse the β-galactosidase activity, cells were centrifuged at 5000×g at 4° C. for 15 minutes. Pellets were re-dissolved in 1% of the original volume using a phosphate buffer B (50 mM Na2HPO4.2H2O, 1 mM MgCl2) and then eight 1250 μl aliquots of each cell-free extract transferred to a deep well plate.

The pellets were subsequently lysed using 0.5 gram silicabeads (0.1 mm) in 800 μl 0.05M NaPi buffer pH=7.0 and 4 repetitions of 30 second bursts in a cell disruptor. The lysed pellets of the same cell-free extract were then recombined in a single 15 ml Geiner-tube. Cultures were centrifuged for 10 minutes at 5000 g after the indicated time-period using a 96-well plate centrifuge. 20 μl of supernatant of the cell lysate was dissolved in 180 μl phosphate buffer A (8.9 gr/I Na2HPO4.2H2O, 6.9 gram/I Na2HPO4.H2O, 1 mM DTT).

Additionally 10, 100 and 100 fold dilutions of the cell lysate phosphate buffer mix were prepared, to which an ONPG stock solution (20 mM in phopshate buffer) at a starting concentration of 1 mM was added. The absorbance at 420 nm was observed over time using a Pharmacia Biotech Ultrospec 2000 UV/visible spectrophotometer using Swift II Application software and the Miller Units were calculated using the above indicated dilutions.

### GOS Synthesis Protocol

Activity was normalized to 2 mM/min in a total volume of 10 ml by dilution using phsopahe buffer B. 15 ml Greiner tubes were pre-warmed which contained 13.5 ml phosphate buffer B at 30°, 50°, and 60° C. The reaction was started by the addition of 1.5 ml cell-free extract (2 mM/min β-galactosidase activity) to the pre-warmed Greiner tubes. The reactions proceeded with a 30 second time interval. 1 ml samples were then transferred to an Eppendorf tube at 0, 30, 60, 90, 120, 180, 240, 300, and 1440 minute intervals. The GOS formation reaction was then stopped by incubation at 100° C. for 5 minutes and the samples immediately stored at −80° C.

Based on the activities of the β-galactosidases found, the actual activity for the GOS formation rate could be predicted. Conversion factors were calculated for each species.

Table 4 below shows the predicted GOS formation rate at 30° C.

TABLE 4

| | | | | | | | Predicted |
|---|---|---|---|---|---|---|---|
| | | | Bgal activity | | | | GOS |
| | | | (Miller Units) | | | | formation |
| | | | Average | Stdev | Used | | rate |
| Strain | | | μmol/min/ | | conversion | Correction | mM/min/100 |
| no. | Species | Subspecies | OD-Unit | | factor | factor | OD units |
| 883 | Streptococcus | | 1960 | 83 | 26.5 | 5.0 | 20.0 |
| | thermophilus | | | | | | |
| 885 | Streptococcus | | 1462 | 103 | 26.5 | 5.0 | 14.9 |
| | thermophilus | | | | | | |
| 882 | Streptococcus | | 1347 | 135 | 26.5 | 5.0 | 13.7 |
| | thermophilus | | | | | | |
| 886 | Streptococcus | | 1291 | 238 | 26.5 | 5.0 | 13.2 |
| | thermophilus | | | | | | |
| 884 | Streptococcus | | 1267 | 86 | 26.5 | 5.0 | 12.9 |
| | thermophilus | | | | | | |
| 114 | Streptococcus | | 1155 | 126 | 26.5 | 5.0 | 11.8 |
| | thermophilus | | | | | | |
| 121 | Streptococcus | | 1140 | 75 | 26.5 | 5.0 | 11.6 |
| | thermophilus | | | | | | |
| 194 | Lactobacillus delbrueckii | bulgaricus | 1019 | 59 | 4.1 | 5.0 | 1.6 |
| 2310 | Streptococcus | | 976 | 68 | 26.5 | 5.0 | 10.0 |
| | thermophilus | | | | | | |
| 106 | Streptococcus | | 954 | 36 | 26.5 | 5.0 | 9.7 |
| | thermophilus | | | | | | |
| 191 | Lactobacillus delbrueckii | bulgaricus | 937 | 62 | 4.1 | 5.0 | 1.5 |
| 2106 | Streptococcus | | 815 | 196 | 26.5 | 5.0 | 8.3 |
| | thermophilus | | | | | | |
| 203 | Lactobacillus delbrueckii | bulgaricus | 804 | 16 | 4.1 | 5.0 | 1.3 |
| 192 | Lactobacillus delbrueckii | bulgaricus | 620 | 320 | 4.1 | 5.0 | 1.0 |
| 195 | Lactobacillus delbrueckii | bulgaricus | 541 | 114 | 4.1 | 5.0 | 0.9 |
| 202 | Lactobacillus delbrueckii | bulgaricus | 540 | 76 | 4.1 | 5.0 | 0.9 |
| 187 | Lactobacillus delbrueckii | bulgaricus | 442 | 61 | 4.1 | 5.0 | 0.7 |
| 189 | Lactobacillus delbrueckii | bulgaricus | 413 | 52 | 4.1 | 5.0 | 0.7 |
| 211 | Lactobacillus helveticus | | 398 | 51 | 4.1 | 5.0 | 0.6 |
| 1456 | Lactobacillus crispatus | | 384 | 6 | 4.1 | 5.0 | 0.6 |
| 204 | Lactobacillus delbrueckii | bulgaricus | 315 | 20 | 4.1 | 5.0 | 0.5 |
| 186 | Lactobacillus delbrueckii | bulgaricus | 282 | 62 | 4.1 | 5.0 | 0.4 |
| 3273 | Lactobacillus helveticus | | 255 | 14 | 4.1 | 5.0 | 0.4 |
| 1795 | Lactobacillus delbrueckii | | 213 | 58 | 4.1 | 5.0 | 0.3 |
| 190 | Lactobacillus delbrueckii | bulgaricus | 150 | | 4.1 | 5.0 | 0.2 |
| 1572 | Lactobacillus fermentum | | 139 | | 4.1 | 5.0 | 0.2 |
| 2954 | Lactobacillus reuteri | | 111 | 0 | 4.1 | 5.0 | 0.2 |
| 3416 | Lactobacillus reuteri | | 105 | 5 | 4.1 | 5.0 | 0.2 |
| 226 | Lactobacillus | | 101 | 7 | 4.1 | 5.0 | 0.2 |
| | acidophilus | | | | | | |
| 3909 | Lactobacillus reuteri | | 94.0 | 1.2 | 4.1 | 5.0 | 0.1 |
| LR92 | L. reuteri | | 90.9 | 6.3 | 4.1 | 5.0 | 0.1 |
| 2955 | Lactobacillus reuteri | | 89.7 | 1.9 | 4.1 | 5.0 | 0.1 |
| LR1 | L. rhamsnosus | | 84.5 | 14.5 | 4.1 | 5.0 | 0.1 |
| LA1 | L. acidophilus | | 72.3 | 6.2 | 4.1 | 5.0 | 0.1 |
| 11796 | Lactobacillus fermentum | | 69.2 | 8.1 | 4.1 | 5.0 | 0.1 |
| D | Lactobacillus | | 41.5 | 1.1 | 4.1 | 5.0 | 0.1 |
| 30226 | Lactobacillus fermentum | | 10.6 | 0.8 | 4.1 | 5.0 | 0.017 |
| C | Lactobacillus | | 1.4 | 0.5 | 4.1 | 5.0 | 0.002 |
| 2828 | Lactobacillus plantarum | | 1.0 | 0.3 | 4.1 | 5.0 | 0.002 |
| 2830 | Lactobacillus plantarum | | 0.6 | 0.0 | 4.1 | 5.0 | 0.001 |
| 2691 | Lactobacillus plantarum | | 0.5 | 0.1 | 4.1 | 5.0 | 0.001 |
| B | Lactobacillus | | 0.4 | 0.2 | 4.1 | 5.0 | 0.001 |
| 101/37 | L. paracasei | | 0.3 | 0.2 | 4.1 | 5.0 | 0.001 |
| 14D | L. plantarum | | 0.3 | 0.0 | 4.1 | 5.0 | 0.000 |
| E | Lactobacillus | | 0.2 | 0.2 | 4.1 | 5.0 | 0.000 |
| A | Lactobacillus | | 0.2 | 0.1 | 4.1 | 5.0 | 0.000 |
| 4204 | Propionibacterium | | 6.0 | 1.5 | 10.8 | 5.0 | 0.025 |
| | acidipropionici | | | | | | |
| 380 | Propionibacterium sp. | | 5.6 | 0.4 | 10.8 | 5.0 | 0.023 |
| 2166 | Propionibacterium | freudenreichii | 4.8 | 0.7 | 10.8 | 5.0 | 0.020 |
| | freudenreichii | | | | | | |
| 359 | Propionibacterium sp. | | 4.5 | 0.1 | 10.8 | 5.0 | 0.019 |
| 1134 | Propionibacterium | shermanii | 4.2 | 0.3 | 10.8 | 5.0 | 0.018 |
| | freudenreichii | | | | | | |
| 364 | Propionibacterium | | 3.7 | 0.4 | 10.8 | 5.0 | 0.015 |
| | jensenii | | | | | | |
| 2060 | Propionibacterium | shermanii | 3.5 | 0.1 | 10.8 | 5.0 | 0.015 |
| | freudenreichii | | | | | | |
| 4199 | Propionibacterium | | 3.5 | 0.1 | 10.8 | 5.0 | 0.015 |
| | acidipropionici | | | | | | |
| 4201 | Propionibacterium | | 3.3 | 0.1 | 10.8 | 5.0 | 0.014 |
| | acidipropionici | | | | | | |
| 2175 | Propionibacterium | freudenreichii | 3.3 | 0.2 | 10.8 | 5.0 | 0.014 |
| | freudenreichii | | | | | | |
| 2145 | Propionibacterium | freudenreichii | 3.3 | 0.2 | 10.8 | 5.0 | 0.014 |
| | freudenreichii | | | | | | |
| 2168 | Propionibacterium | freudenreichii | 3.1 | 0.6 | 10.8 | 5.0 | 0.013 |
| | freudenreichii | | | | | | |
| 2174 | Propionibacterium | freudenreichii | 3.1 | 0.6 | 10.8 | 5.0 | 0.013 |
| | freudenreichii | | | | | | |

Table 5 below shows the predicted GOS formation rate at 50° C.

TABLE 5

| | | | | | | | Predicted |
|---|---|---|---|---|---|---|---|
| | | | Bgal activity | | | | GOS |
| | | | (Miller Units) | | | | formation |
| | | | Average | Stdev | Used | | rate |
| Strain | | | μmol/min/ | | conversion | Correction | mM/min/100 |
| no. | Species | Subspecies | OD-Unit | | factor | factor | OD units |
| 883 | Streptococcus | | 1960 | 83 | 26.5 | 5.0 | 20.0 |
| | thermophilus | | | | | | |
| 885 | Streptococcus | | 1462 | 103 | 26.5 | 5.0 | 14.9 |
| | thermophilus | | | | | | |
| 882 | Streptococcus | | 1347 | 135 | 26.5 | 5.0 | 13.7 |
| | thermophilus | | | | | | |
| 886 | Streptococcus | | 1291 | 238 | 26.5 | 5.0 | 13.2 |
| | thermophilus | | | | | | |
| 884 | Streptococcus | | 1267 | 86 | 26.5 | 5.0 | 12.9 |
| | thermophilus | | | | | | |
| 114 | Streptococcus | | 1155 | 126 | 26.5 | 5.0 | 11.8 |
| | thermophilus | | | | | | |
| 121 | Streptococcus | | 1140 | 75 | 26.5 | 5.0 | 11.6 |
| | thermophilus | | | | | | |
| 194 | Lactobacillus delbrueckii | bulgaricus | 1019 | 59 | 4.1 | 5.0 | 1.6 |
| 2310 | Streptococcus | | 976 | 68 | 26.5 | 5.0 | 10.0 |
| | thermophilus | | | | | | |
| 106 | Streptococcus | | 954 | 36 | 26.5 | 5.0 | 9.7 |
| | thermophilus | | | | | | |
| 191 | Lactobacillus delbrueckii | bulgaricus | 937 | 62 | 4.1 | 5.0 | 1.5 |
| 2106 | Streptococcus | | 815 | 196 | 26.5 | 5.0 | 8.3 |
| | thermophilus | | | | | | |
| 203 | Lactobacillus delbrueckii | bulgaricus | 804 | 16 | 4.1 | 5.0 | 1.3 |
| 192 | Lactobacillus delbrueckii | bulgaricus | 620 | 320 | 4.1 | 5.0 | 1.0 |
| 195 | Lactobacillus delbrueckii | bulgaricus | 541 | 114 | 4.1 | 5.0 | 0.9 |
| 202 | Lactobacillus delbrueckii | bulgaricus | 540 | 76 | 4.1 | 5.0 | 0.9 |
| 187 | Lactobacillus delbrueckii | bulgaricus | 442 | 61 | 4.1 | 5.0 | 0.7 |
| 189 | Lactobacillus delbrueckii | bulgaricus | 413 | 52 | 4.1 | 5.0 | 0.7 |
| 211 | Lactobacillus helveticus | | 398 | 51 | 4.1 | 5.0 | 0.6 |
| 1456 | Lactobacillus crispatus | | 384 | 6 | 4.1 | 5.0 | 0.6 |
| 204 | Lactobacillus delbrueckii | bulgaricus | 315 | 20 | 4.1 | 5.0 | 0.5 |
| 186 | Lactobacillus delbrueckii | bulgaricus | 282 | 62 | 4.1 | 5.0 | 0.4 |
| 3273 | Lactobacillus helveticus | | 255 | 14 | 4.1 | 5.0 | 0.4 |
| 1795 | Lactobacillus delbrueckii | | 213 | 58 | 4.1 | 5.0 | 0.3 |
| 190 | Lactobacillus delbrueckii | bulgaricus | 150 | | 4.1 | 5.0 | 0.2 |
| 1572 | Lactobacillus fermentum | | 139 | | 4.1 | 5.0 | 0.2 |
| 2954 | Lactobacillus reuteri | | 111 | 0 | 4.1 | 5.0 | 0.2 |
| 3416 | Lactobacillus reuteri | | 105 | 5 | 4.1 | 5.0 | 0.2 |
| 226 | Lactobacillus | | 101 | 7 | 4.1 | 5.0 | 0.2 |
| | acidophilus | | | | | | |
| 3909 | Lactobacillus reuteri | | 94.0 | 1.2 | 4.1 | 5.0 | 0.1 |
| LR92 | L. reuteri | | 90.9 | 6.3 | 4.1 | 5.0 | 0.1 |
| 2955 | Lactobacillus reuteri | | 89.7 | 1.9 | 4.1 | 5.0 | 0.1 |
| LR1 | L. rhamsnosus | | 84.5 | 14.5 | 4.1 | 5.0 | 0.1 |
| LA1 | L. acidophilus | | 72.3 | 6.2 | 4.1 | 5.0 | 0.1 |
| 11796 | Lactobacillus fermentum | | 69.2 | 8.1 | 4.1 | 5.0 | 0.1 |
| D | Lactobacillus | | 41.5 | 1.1 | 4.1 | 5.0 | 0.1 |
| 30226 | Lactobacillus fermentum | | 10.6 | 0.8 | 4.1 | 5.0 | 0.017 |
| C | Lactobacillus | | 1.4 | 0.5 | 4.1 | 5.0 | 0.002 |
| 2828 | Lactobacillus plantarum | | 1.0 | 0.3 | 4.1 | 5.0 | 0.002 |
| 2830 | Lactobacillus plantarum | | 0.6 | 0.0 | 4.1 | 5.0 | 0.001 |
| 2691 | Lactobacillus plantarum | | 0.5 | 0.1 | 4.1 | 5.0 | 0.001 |
| B | Lactobacillus | | 0.4 | 0.2 | 4.1 | 5.0 | 0.001 |
| 101/37 | L. paracasei | | 0.3 | 0.2 | 4.1 | 5.0 | 0.001 |
| 14D | L. plantarum | | 0.3 | 0.0 | 4.1 | 5.0 | 0.000 |
| E | Lactobacillus | | 0.2 | 0.2 | 4.1 | 5.0 | 0.000 |
| A | Lactobacillus | | 0.2 | 0.1 | 4.1 | 5.0 | 0.000 |
| 4204 | Propionibacterium | | 6.0 | 1.5 | 10.8 | 5.0 | 0.025 |
| | acidipropionici | | | | | | |
| 380 | Propionibacterium sp. | | 5.6 | 0.4 | 10.8 | 5.0 | 0.023 |
| 2166 | Propionibacterium | freudenreichii | 4.8 | 0.7 | 10.8 | 5.0 | 0.020 |
| | freudenreichii | | | | | | |
| 359 | Propionibacterium sp. | | 4.5 | 0.1 | 10.8 | 5.0 | 0.019 |
| 1134 | Propionibacterium | shermanii | 4.2 | 0.3 | 10.8 | 5.0 | 0.018 |
| | freudenreichii | | | | | | |
| 364 | Propionibacterium | | 3.7 | 0.4 | 10.8 | 5.0 | 0.015 |
| | jensenii | | | | | | |
| 2060 | Propionibacterium | shermanii | 3.5 | 0.1 | 10.8 | 5.0 | 0.015 |
| | freudenreichii | | | | | | |
| 4199 | Propionibacterium | | 3.5 | 0.1 | 10.8 | 5.0 | 0.015 |
| | acidipropionici | | | | | | |
| 4201 | Propionibacterium | | 3.3 | 0.1 | 10.8 | 5.0 | 0.014 |
| | acidipropionici | | | | | | |
| 2175 | Propionibacterium | freudenreichii | 3.3 | 0.2 | 10.8 | 5.0 | 0.014 |
| | freudenreichii | | | | | | |
| 2145 | Propionibacterium | freudenreichii | 3.3 | 0.2 | 10.8 | 5.0 | 0.014 |
| | freudenreichii | | | | | | |
| 2168 | Propionibacterium | freudenreichii | 3.1 | 0.6 | 10.8 | 5.0 | 0.013 |
| | freudenreichii | | | | | | |
| 2174 | Propionibacterium | freudenreichii | 3.1 | 0.6 | 10.8 | 5.0 | 0.013 |
| | freudenreichii | | | | | | |

### GOS Analysis Protocol

High-Performance Anion-Exchange Chromatography with Pulsed Amperometric Detection (HPAEC-PAD) was used to undertake the GOS analysis. HPAEC-PAD analyses were performed on a DX-500 BIO-LCsystem (Dionex) equipped with a PAD. Galactooligosaccharide fractions were separated on CarboPac PA1 column with dimensions 250 mm*4 mm t a flow rate of 1 mL/min at 22° C. A CarboPac PA1 guard column with dimensions 50*4 mm i.d. (Dionex) was used for column protection. The eluents used for the analysis were (A) 500 mM NaOAc+100 mMNaOH, (B) 100 mMNaOH and (C) Milli-Q water.

Eluents A and B were mixed to form the following gradient: 100% B from 0 to 5 min followed by 0-26% A in 73 min. After each run, the column was washed with 100% A for 6 min and re-equilibrated for 10 min at 100% B. Peak identification occurred on the basis of comparison of peak distribution of the HPLC chromatogram described in J. Agric. Food Chem. 2009, 57, 8488-8495. Lactose was used as a standard for elution time normalization.

### Results

To determine the ratio between highly formed GOS species the most prevalent GOS species for P. jensenii were quantified and the ratio between the two species calculated at different temperatures and time points. As shown in Table 6 below and illustrated in FIG. 1, it was found that the species ratio showed a strong temperature dependence and a small time dependence.

TABLE 6

| | Expected GOS | Expected GOS | β-D-Gal- | |
|---|---|---|---|---|
| | linkage type | linkage type | (1f4)-β-D-Gal- | Ratio |
| Strain | Temp | Unknown | (1f4)-D-Glc | 2:1 |
| Propionibacterium | Temp = 30 C., | 0.2 | 4.2 | 18.3 |
| jensenii | Time 5 H | | | |
| Propionibacterium | Temp = 50 C., | 1.2 | 2.9 | 2.5 |
| jensenii | Time 5 H | | | |
| Propionibacterium | Temp = 30 C., | 1.0 | 12.0 | 11.8 |
| jensenii | Time 24 H | | | |
| Propionibacterium | Temp = 50 C., | 4.4 | 6.2 | 1.4 |
| jensenii | Time 24 H | | | |

Based on standard thermodynamics it was assumed that at 50° C. the β-galactosidase reaction occurs at a 4-8 times higher rate than at 30° C. For tested samples where the GOS formation rate was at a stage where this was expected to be linear the GOS formation rates were plotted. As shown in Table 7 below and illustrated in FIG. 2, the two Lactobacillus strains showed a 3-4 fold in GOS formation rate at 50° C., for both Propionibacterium strains no significant increase in activity was detected.

TABLE 7

| Strain | Temp | Total GOS |
|---|---|---|
| L. reuteri | 30° C. | 3.7 |
| | 50° C. | 13.9 |
| L. fermentum | 30° C. | 1.4 |
| | 50° C. | 4.1 |
| P. jensenii | 30° C. | 4.5 |
| | 50° C. | 4.1 |
| P. freudenreichii | 30° C. | 5.3 |
| | 50° C. | 6.1 |

The theoretical GOS formation rate was calculated based on the β-galactosidase activity, expressed in Miller Units, measured in Phase 2 of the study. Table 8 below shows the ratio of actual GOS formation rate over theoretical GOS formation rate and FIG. 3 shows this plotted for both 30° C. and 50° C. Surprisingly, and advantageously, GOS formation rates were always found to be higher than the theoretical GOS formation rates.

TABLE 8

| | | Actual/Theoretical | Actual/Theoretical | |
|---|---|---|---|---|
| Strain | No | GOS (30 C.) | GOS (50 C.) | Ratio |
| S. thermophilus | 883 | | 28.3 | |
| S. thermophilus | 883 | | 11.3 | |
| S. thermophilus | 114 | | 39.9 | |
| L. helveticus | 211 | 1.4 | | |
| L. delbrueckii | 191 | 3.1 | | |
| L. reuteri | 2954 | 1.5 | 5.5 | 3.8 |
| L. fermentum | 11796 | 0.9 | 2.7 | 2.9 |
| P. jensenii | 364 | 13.6 | 12.5 | 0.9 |
| P. freudenreichii | 1134 | 7.9 | 9.1 | 1.2 |
| Average | | | 26.5 | |
| streptococci | | | | |
| Average lactobacilli | 1.7 | 4.1 | 2.4 | |
| Average propioni's | 10.8 | 10.8 | 1.0 | |

The β-galactosidase activity analysed in the initial phase of experiments in general appeared to be higher than those activities determined in the later phase. To find out whether there is a consistent error in the methodology the ratios of the activities in phase 1 and 2 were calculated (and shown in Table 9 below) and plotted on a graph shown in FIG. 4. FIG. 4 shows that for most samples a 5-fold difference is detected. Some samples clearly show much higher differences, and this is expected that these differences are mainly due to the differences in the growth phase of the cells.

TABLE 9

| Strain no | Growth Phase | Ratio Phase 1/Phase 2 |
|---|---|---|
| 883 | Mid-log | 6.7 |
| 883 | Stationary | 4.5 |
| 114 | Mid-log | 27.7 |
| 114 | Stationary | 16.0 |
| 211 | Mid-log | 1.8 |
| 211 | Stationary | Very High |
| 191 | Mid-log | 9.1 |
| 191 | Stationary | Very High |
| 2954 | Mid-log | 5.1 |
| 2954 | Mid-log | 5.1 |
| 2954 | Stationary | 6.2 |
| 11796 | Mid-log | 3.8 |
| 11796 | Mid-log | 3.8 |
| 11796 | Stationary | 21.4 |
| 364 | Mid-log | 7.9 |
| 364 | Mid-log | 7.9 |
| 364 | Stationary | 14.4 |
| 1134 | Mid-log | 1.5 |
| 1134 | Stationary | 5.0 |
| 1134 | Stationary | 5.0 |
| 4204 | Mid-log | 1.2 |
| 4204 | Stationary | 6.1 |

To assess whether the expression of β-galactosidase was dependent on the growth phase of the organism, the activity (as measured in Miller Units) was plotted for all strains. Table 10 and FIG. 5 show the data and plot respectively. It was found that for most strains a Log:Stat ratio 1 was found indicating that the activity of β-galactosidase is higher in the Log phase than in the stationary phase. With a few exceptions (strain 211 and 191) these differences are limited and it may be that the higher biomass yield in stationary phase off-sets the lower β-galactosidase activities.

TABLE 10

| | | Ratio Log/Stat |
|---|---|---|
| Streptococcus thermophilus | 883 | 0.7 |
| Streptococcus thermophilus | 114 | 0.6 |
| Lactobacillus helveticus | 211 | Very high |
| Lactobacillus delbrueckii | 191 | 12.5 |
| Lactobacillus reuteri | 2954 | 1.2 |
| Lactobacillus fermentum | 11796 | 5.6 |
| Propionibacterium jensenii | 364 | 1.8 |
| Propionibacterium freudenreichii | 1134 | 3.4 |
| Propionibacterium acidipropionici | 4204 | 4.9 |

### CONCLUSIONS

GOS formation rates for the strains selected on the basis of the Miller Unit value were deemed as a good predictor for those strains showing good GOS production even when grown in non-optimised conditions. It was established that all Lactobacillus strains that gave β-galactosidase activity above 60 Miller Units in phase 1 produced GOS in the phase 2 feasibility study, whereas the one control strain that was below the 60 Miller Unit cut-off did not. Most of S. thermophilus showed Bgal activities significantly higher than 60 miller units and only those which appeared to be the best were selected for taking further to the phase 2 feasibility study. For Propionibacterium all were below the 60 miller unit cut off, but all strains selected produced GOS.

In general GOS formation rates were 3-4 fold higher at 50° C. as compared to 30° C. for the lactobacilli strains. The Propionibacterium cell-free extracts showed approximately similar GOS formation rates at 30° C. and 50° C. All samples show a different GOS profile than the GOS produced by Apergillus Oryzea enzyme. Specifically strain 364 (P. jensenii) showed significant GOS production. The studies established that Miller Unit activities translated well to potential GOS activity and proved to be a useful and accurate predictor of GOS production. For specific cases GOS production was up to 15 fold higher than ONPG hydrolysis activity had initially suggested. In general, the later GOS synthesis phase showed a 5-fold lower β-galactosidase activities as compared to the initial screening phase.

Using the described screening method, and Miller Unit cut-off of 60 (for Streptococcus or Lactobacillus) and 3 (for Propionibacterium), allows for a quick and reliable prediction of the likelihood of whether a bacteria can produce GOS in sufficient yields so as to allow purification and further testing of its prebiotic properties in vitro. It can also be used to help identify any potentially novel GOS structures. It advantageously provides a systematic methodology which permits screening of large numbers of bacteria for the potential to produce GOS, identify novel GOS, and scale up to test in in vitro models. Furthermore, as the Miller Unit is a composite of the growth rate, enzyme production and activity, then this parameter enables focus on only those strains which are most likely to be commercially viable.

The forgoing embodiments are not intended to limit the scope of the protection afforded by the claims, but rather to describe examples of how the invention may be put into practice.

1. A method of screening a Streptococcus, Lactobacillus or Propionibacterium bacterial strain for galactooligosaccharides (GOS) yield comprising assessing β-galactosidase activity of the GOS produced by the strain under growth conditions and selecting a bacterial strain producing GOS having β-galactosidase activity of:
a) Miller Units equal to or greater than about 60 for a Streptococcus or Lactobacillus strain; or
b) Miller Units equal to or greater than about 3 for a Propionibacterium strain.

2. The method as claimed in claim 1, wherein the growth conditions comprise growing the bacterial strain for a given incubation time, lysing the bacterial cells to provide a lysate and assessing the β-galactosidase activity in the lysate.

3. The method as claimed in claim 1, wherein assessing β-galactosidase activity comprises:
i) incubating the bacterial strain at about 37° C. for up to 40 hours;
ii) centrifuging the bacterial cells at a temperature lower than about 37° C.;
iii) lysing the cells and removing a supernatant from the lysed cells; and
iv) assessing β-galactosidase activity, expressed in Miller Units, in the supernatant.

4. The method as claimed in claim 1, further comprising:
assessing β-galactosidase activity of GOS produced by the selected bacterial strain at a higher temperature than about 37° C. and a lower temperature than about 37° C. and identifying the selected bacterial strain having the highest β-galactosidase activity as a strain with highest yield of GOS.

5. The method as claimed in claim 4, wherein the higher temperature is about 50° C. and the lower temperature is about 30° C.

6. The method as claimed in claim 1, wherein the Lactobacillus bacterial strain is selected from the group consisting of: Lactobacillus acidophilus, Lactobacillus reuteri, Lactobacillus rhamnosus, Lactobacillus plantarum, Lactobacillus delbrueckii, Lactobacillus fermenturn, Lactobacillus crispatus, Lactobacillus buchneri Lactobacillus helveticus, Streptococcus thermophilus, Propionibacterium jensenii; Propionibacterium freudenreichii; and Propionibacterium acidipropionici.

7. A prebiotic composition comprising a galactooligosaccharide (GOS) produced by a Streptococcus, Lactobacillus or Propionibacterium bacterial strain, wherein the GOS has a high β-galactosidase activity, and wherein the high β-galactosidase activity is:
a) a Miller Unit equal to or greater than about 60 for a Streptococcus or Lactobacillus strain; or
b) a Miller Unit equal to or greater than about 3 for a Propionibacterium strain.

8. The prebiotic composition as claimed in claim 7, wherein the GOS is produced and/or is selective for one of more of the following bacterial strains: Lactobacillus acidophilus, Lactobacillus reuteri, Lactobacillus rhamnosus, Lactobacillus plantarum, Lactobacillus delbrueckii, Lactobacillus fermentum, Lactobacillus crispatus, Lactobacillus buchneri Lactobacillus helveticus, Streptococcus thermophilus, Propionibacterium jensenii; Propionibacterium freudenreichii; Propionibacterium acidipropionici, sub-species thereof or mutant strain thereof.

9. The prebiotic composition as claimed in claim 7, wherein the composition is encapsulated.

10. The prebiotic composition as claimed in claim 7 further comprising an excipient or carrier compound, the excipient or carrier compound providing for the prebiotic composition to pass through a gastrointestinal environment with retained functional properties.

11. The prebiotic composition as claimed in claim 7, wherein the composition is a liquid, a powder or a form that can be mixed with a solid or liquid food stuff.

12. The prebiotic composition as claimed in claim 7, wherein the GOS is produced by a bacterial strain identified in the screening method of claim 1.

13. The method of claim 1 wherein the Lactobacillus bacterial strain is: Lactobacillus acidophilus, Lactobacillus reuteri, Lactobacillus rhamnosus, Lactobacillus plantarum, Lactobacillus delbrueckii, Lactobacillus fermentum, Lactobacillus crispatus, Lactobacillus buchneri Lactobacillus helveticus, Streptococcus thermophilus, Propionibacterium jensenii; Propionibacterium freudenreichii; Propionibacterium acidipropionici, a sub-species thereof or mutant strain thereof.

14. The method of claim 2 wherein the Lactobacillus bacterial strain is: Lactobacillus acidophilus, Lactobacillus reuteri, Lactobacillus rhamnosus, Lactobacillus plantarum, Lactobacillus delbrueckii, Lactobacillus fermentum, Lactobacillus crispatus, Lactobacillus buchneri Lactobacillus helveticus, Streptococcus thermophilus, Propionibacterium jensenii; Propionibacterium freudenreichii; Propionibacterium acidipropionici, a sub-species thereof or mutant strain thereof.

15. The method of claim 4 wherein the Lactobacillus bacterial strain is: Lactobacillus acidophilus, Lactobacillus reuteri, Lactobacillus rhamnosus, Lactobacillus plantarum, Lactobacillus delbrueckii, Lactobacillus fermenturn, Lactobacillus crispatus, Lactobacillus buchneri Lactobacillus helveticus, Streptococcus thermophilus, Propionibacterium jensenii; Propionibacterium freudenreichii; Propionibacterium acidipropionici, a sub-species thereof or a mutant strain thereof.

16. A method of selecting a Streptococcus, Lactobacillus or Propionibacterium bacterial strain that produces a high yield of galactooligosaccharides (GOS) comprising:
incubating the bacterial strain under appropriate conditions for production of galactooligosacccharide (GOS);
assessing the GOS for β-galactosidase activity; and
selecting bacterial strains that produce GOS having a higher β-gactosidase activity, wherein a higher β-galactosidase activity is:
a) Miller Units equal to or greater than about 60 for a Streptococcus or Lactobacillus strain; or
b) Miller Units equal to or greater than about 3 for a Propionibacterium strain.

17. The method of claim 16 wherein the GOS is in substantially the same form as GOS produced by a reverse β-galactosidase reaction in the selected bacterial strain.

18. The method of claim 17 wherein the selected bacterial strain is Propionibacterium jensenii.

19. The method of claim 16 wherein a selected bacterial strain producing a galactooligosaccharides (GOS) having a higher β-gactosidase activity has a higher galactooligosaccharides (GOS) yield compared to a bacterial strain producing a galactooligosaccharides (GOS) having a lower β-gactosidase activity.

20. The method of claim 19 wherein a lower GOS producing bacterial strain is a bacterial strain producing a GOS having a lower β-gactosidase activity of:
a) Miller Units less than about 60 for a Streptococcus or Lactobacillus strain; or
b) Miller Units less than about 3 for a Propionibacterium strain.
